(19) United States
(12) Patent Application Publication
(10) Pub. No.: US 2021/0074406 A1
(43) Pub. Date: Mar. 11, 2021

(54) PRE-THERAPEUTIC AND THERAPEUTIC DIGITAL MEDICAL DEVICE AND METHOD

(71) Applicant: Blue Note Therapeutics, Inc., San Francisco, CA (US)

(72) Inventors: Geoffrey S. EICH, Camarillo, CA (US); Michael MCKINLEY, Camarillo, CA (US); Richard LIT, Malibu, CA (US); Michael MALECKI, Westlake Village, CA (US); Mark ELFERS, Simi Valley, CA (US); Sadie WHITTAKER, Los Angeles, CA (US)

(21) Appl. No.: 17/014,749

(22) Filed: Sep. 8, 2020

Related U.S. Application Data
(60) Provisional application No. 62/897,132, filed on Sep. 6, 2019.

(51) Int. Cl. G16H 20/70; G16H 50/30; G16H 10/60; G16H 50/20; G16H 15/00; G16H 10/20; G16H 80/00; G16H 40/67; G16H 40/20; A61B 5/00; A61B 5/16; G09B 19/00; H04L 29/08

(52) U.S. Cl. CPC G16H 20/70; G16H 50/30; G16H 10/60; G16H 50/20; G16H 15/00; G16H 10/20; H04L 67/12; G16H 40/67; G16H 40/20; A61B 5/4809; A61B 5/165; A61B 5/7475; G09B 19/00; G16H 80/00

(57) ABSTRACT

A method, medium and system for pre-therapeutic processing. The method, medium and system may establish a baseline psychosocial wellness score for a patient. The method, medium and system may establish an interface between the patient and a digital therapist. The method, medium and system may introduce, reinforce, model and instill adaptation skills to overcome adjustment or depressive disorders. The method, medium and system may identify changes from the baseline. The method, medium and system may determine whether a communication initiation threshold is met. The method, medium and system may receive an election to participate in assessment from the patient. The method, medium and system may format patient data, including the changes from the baseline, and assessment for submission. The method, medium and system may submit patient data to a healthcare provider (“HCP”). Further, the method, medium and system may establish a communication between the patient and the HCP.

### CROSS-REFERENCE TO RELATED APPLICATION

This application claims the benefit of U.S. Provisional Patent Application No. 62/897,132 filed on Sep. 6, 2019. The contents of the prior application is incorporated herein by reference in the entirety.

### TECHNICAL FIELD

Aspects of the present disclosure are directed to pre-therapeutic and therapeutic digital medical devices and methods. Specifically, aspects of the present disclosure are directed to pre-therapeutic and therapeutic processing, diagnosis, and treatment of depression, distress, anxiety, fear of recurrence, fatigue, pain, inflammation, sexual dysfunction, complications of stem cell transplant, and insomnia and other conditions and symptoms in patients suffering from cancer, among other diseases that have the potential of significantly affecting patients' health.

### BACKGROUND

A typical and prevalent side effect of a cancer diagnosis, and/or a diagnosis of another serious illness, is the advent of depression and other related symptoms and conditions, such as distress, anxiety, fear of recurrence, fatigue, and insomnia, among others. Often, however, the main focus is on the diagnosis and treatment of the cancer or other serious illness, and the symptoms of depression and related conditions often go unnoticed or ignored. There is an unmet need in the art, therefore, for pre-therapeutic and therapeutic methods and systems that allow pre-therapeutic processing, diagnosis, and treatment of depression and other conditions and symptoms in patients suffering from cancer and other serious medical conditions.

### SUMMARY

The following presents a simplified summary of one or more aspects of the disclosure in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects of the disclosure in a simplified form as a prelude to the more detailed description that is presented later.

According to some aspects, the present disclosure is directed to pre-therapeutic and therapeutic methods and systems that allow pre-therapeutic processing, diagnosis, and treatment of depression and other conditions and symptoms in patients suffering from cancer and other serious medical conditions. According to some aspects, the present disclosure is directed to pre-therapeutic and therapeutic methods and systems that have the capability to influence patients' neurobiological, biochemical or epigenetic markers, and can measure changes in the neurobiological, biochemical or epigenetic markers. In some variations, the present disclosure is directed to pre-therapeutic and therapeutic methods and systems that provide confidential disease state education for patients.

To the accomplishment of the foregoing and related ends, the one or more aspects of the disclosure comprise the features hereinafter fully described and particularly pointed out in the claims. The following description and the annexed drawings set forth in detail certain illustrative features of the one or more aspects. These features are indicative, however, of but a few of the various ways in which the principles of various aspects can be employed, and this description is intended to include all such aspects and their equivalents.

### BRIEF DESCRIPTION OF THE DRAWINGS

The novel features believed to be characteristic of aspects described herein are set forth in the appended claims. In the descriptions that follow, like parts are marked throughout the specification and drawings with the same numerals, respectively. The drawing figures are not necessarily drawn to scale and certain figures can be shown in exaggerated or generalized form in the interest of clarity and conciseness. The disclosure itself, however, as well as a preferred mode of use, further objects and advances thereof, will be best understood by reference to the following detailed description of illustrative embodiments when read in conjunction with the accompanying drawings, wherein:

FIGS. 1A-1B illustrate a flowchart showing an example method for pre-therapeutic and therapeutic processing and treatment, according to an aspect of the disclosure;

FIGS. 2A-2B illustrate a flowchart showing an example method for therapeutic processing and treatment, according to an aspect of the disclosure;

FIG. 3 presents an example system diagram of various hardware components and other features for use in accordance with aspects of the disclosure;

FIG. 4 is a block diagram of various example system components for use in accordance with aspects of the disclosure;

FIGS. 5A-5C illustrate an example Beck-Depression Inventory (BDI);

FIGS. 6A-6B illustrate an example Cancer Coping Questionnaire (CCQ);

FIG. 7 illustrates an example Center for Epidemiologic Studies Depression Scale (CES-D);

FIG. 8 illustrates an example Hospital Anxiety and Depression Scale (HADS);

FIGS. 9A-9B illustrate an example Hamilton Depression Rating Scale (HAM-D);

FIGS. 10A-10B illustrate an example Harvard Department of Psychiatry National Depression Screening Day Scale (HANDS);

FIGS. 11A-11C illustrate an example Mental Adjustment to Cancer (MACS) Scale;

FIGS. 12A-12B illustrate an example State-Trait Anxiety Inventory for Adults (MACS) Scale;

FIGS. 13A-13V illustrate example graphical user interface (GUI) layouts for a pre-therapeutic and therapeutic processing and treatment, according to an aspect of the disclosure system; and

FIGS. 14A-14C presents an example system backend schematic for pre-therapeutic and therapeutic processing and treatment, according to an aspect of the disclosure.

### DETAILED DESCRIPTION

The following includes definitions of selected terms employed herein. The definitions include various examples and/or forms of components that fall within the scope of a term and that can be used for implementation. The examples are not intended to be limiting.

The term “bus,” as used herein, can refer to an interconnected architecture that is operably connected to transfer data between computer components within a singular or multiple systems. The bus can be a memory bus, a memory controller, a peripheral bus, an external bus, a crossbar switch, and/or a local bus, among others. The bus can also be a vehicle bus that interconnects components inside a vehicle using protocols such as Controller Area Network (CAN), Local Interconnect Network (LIN), among others.

The term “location,” as used herein, can refer to a position of an object in space. A location can be indicated using a coordinate system. For example, a location can be represented as a longitude and latitude. In another aspect, a location can include a height. Moreover, in an example, the location can be relative to an object, such as a device detecting location of another device, and the location can be indicated based on the device detecting the location.

The term “memory,” as used herein, can include volatile memory and/or nonvolatile memory. Non-volatile memory can include, for example, ROM (read only memory), PROM (programmable read only memory), EPROM (erasable PROM) and EEPROM (electrically erasable PROM). Volatile memory can include, for example, RAM (random access memory), synchronous RAM (SRAM), dynamic RAM (DRAM), synchronous DRAM (SDRAM), double data rate SDRAM (DDR SDRAM), and direct RAM bus RAM (DRRAM).

The term “operable connection,” as used herein, can include a connection by which entities are “operably connected,” is one in which signals, physical communications, and/or logical communications can be sent and/or received. An operable connection can include a physical interface, a data interface and/or an electrical interface.

The term “processor,” as used herein, can refer to a device that processes signals and performs general computing and arithmetic functions. Signals processed by the processor can include digital signals, data signals, computer instructions, processor instructions, messages, a bit, a bit stream, or other computing that can be received, transmitted and/or detected. A processor, for example, can include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described herein.

The detailed description set forth below in connection with the appended drawings is intended as a description of various configurations and is not intended to represent the only configurations in which the concepts described herein can be practiced. The detailed description includes specific details for the purpose of providing a thorough understanding of various concepts. However, it will be apparent to those skilled in the art that these concepts can be practiced without these specific details. In some instances, well known structures and components are shown in block diagram form in order to avoid obscuring such concepts.

Several aspects of certain systems will now be presented with reference to various example systems and methods. These systems and methods will be described in the following detailed description and illustrated in the accompanying drawings by various blocks, modules, components, circuits, steps, processes, algorithms, etc. (collectively referred to as “elements”). These elements can be implemented using electronic hardware, computer software, or any combination thereof. Whether such elements are implemented as hardware or software depends upon the particular application and design constraints imposed on the overall system.

By way of example, an element, or any portion of an element, or any combination of elements can be implemented with a “processing system” that includes one or more processors. One or more processors in the processing system can execute software. Software shall be construed broadly to mean instructions, instruction sets, code, code segments, program code, programs, subprograms, software modules, applications, software applications, software packages, routines, subroutines, objects, executables, threads of execution, procedures, functions, whether referred to as software, firmware, middleware, microcode, hardware description language, or otherwise.

Accordingly, in one or more aspects, the functions described can be implemented in hardware, software, firmware, or any combination thereof. If implemented in software, the functions can be stored on or encoded as one or more instructions or code on a computer-readable medium. Computer-readable media includes computer storage media. Storage media can be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer.

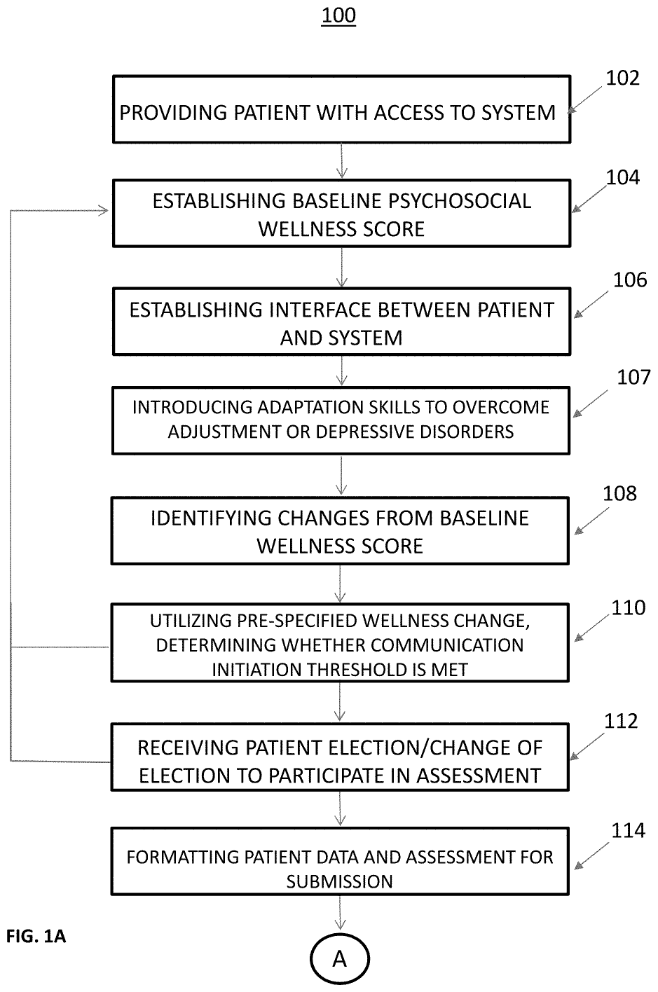

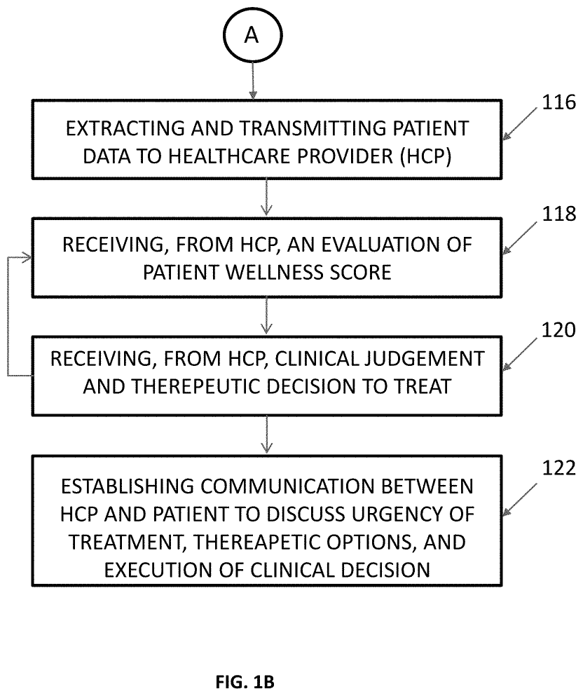

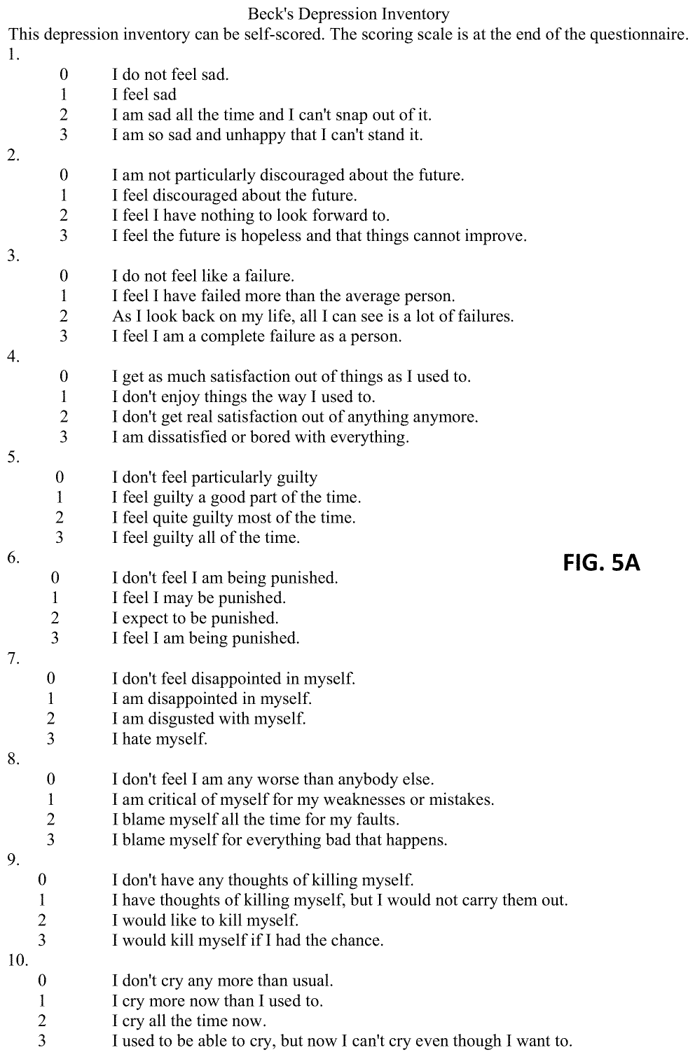

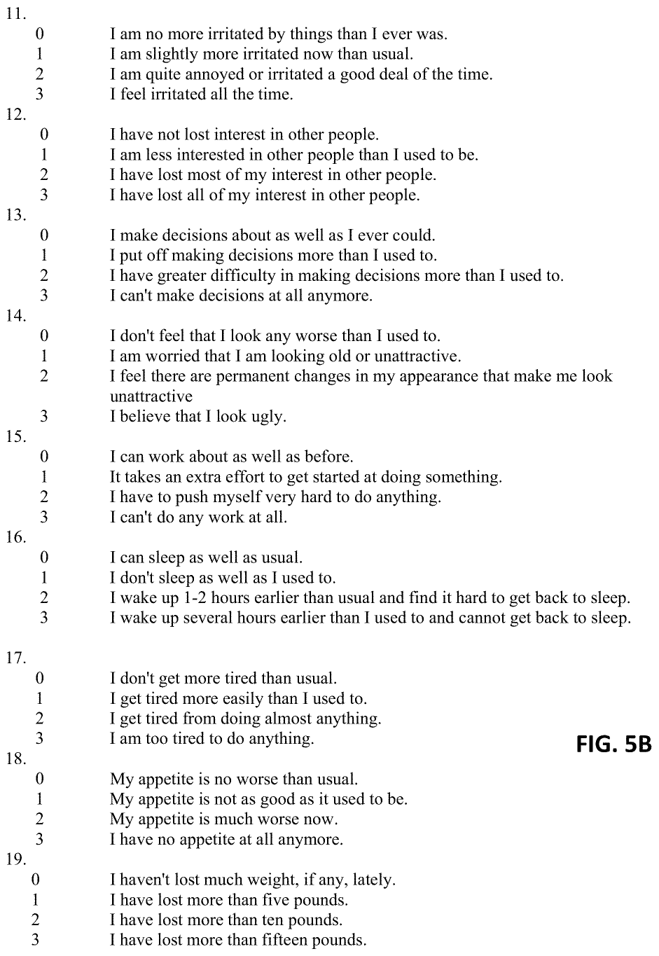

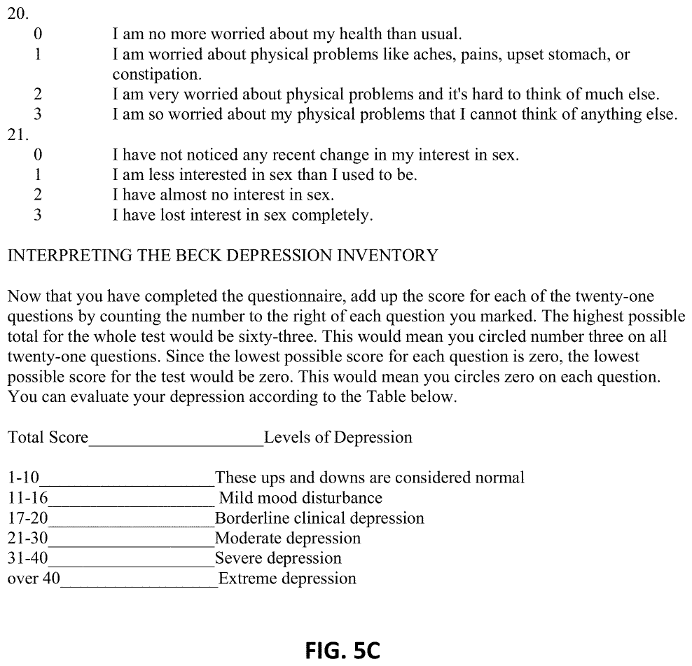

Referring to FIGS. 1A-1B, therein shown is a flowchart showing an example method 100 for pre-therapeutic processing and execution, according to an aspect of the disclosure. In one example, one of the early decisions made by a healthcare provider team (HCP), including, e.g., oncologist, oncology clinic, oncology social worker, advanced practice nurse, nurse practitioner, practicing in hospital, medical practice, or outpatient clinic settings, after a patient is diagnosed with cancer or other serious disease is to provide the patient with access to the system for pre-therapeutic processing and management of depression and other conditions and symptoms. In one variation, the HCP may, in view of the patient's history, make a decision to provide the patient with access to the system for pre-therapeutic processing and management of depression and other conditions and symptoms. In one variation, the patient may be able to access the system for pre-therapeutic processing and management of depression after conducting an Internet search related to the patient's symptoms or disease. In accordance with some aspects, the system for pre-therapeutic processing and management may be a tool that the patient is able to use, designed to create longitudinal data and a reference baseline of the patient's psychosocial wellness. The patient may start to use the tool prior to the cancer (or other serious disease) diagnosis all the way through remission and survivorship. At block 102, in accordance with one aspect, the physician may introduce the patient to the tool and its purposes, one of which may be to enable the patient to have access to important longitudinal data, or data that the patient reports over time, for example. At block 104, a baseline psychosocial wellness score may be established. Further, one of the goals of the tool may be to enable each HCP to select the appropriate accepted screening tool that may be suitable for their practice. In accordance with one aspect, it may be desirable to measure depression and distress in oncology patients regularly, either annually or bi-annually, for example. There are a number of accepted screening tools, including the Beck-Depression Inventory (BDI), the National Comprehensive Cancer Network (NCCN) Distress Thermometer, Cancer Coping Questionnaire (CCQ), Cancer Support Source™ (CSS), Hamilton Anxiety Rating Scale (HAM-A), Hamilton Depression Rating Scale (HAM-D), Patient-Reported Outcomes Measurement Information System (PROMIS) 57, Measure of Current Status (MOCS), Intrusive Events Scale (IES), Patient Activation Measure (PAM), among others, that may be used for screening patients in an oncology setting. In some situations, the preferred inventory or screening tool may be consistent with those used in other settings of care or for other diseases, such as the Hospital Anxiety and Depression Scale (HADS). Most accepted screening tools include qualitative questionnaires with an aggregate scoring methodology. Examples of various features of accepted screening tools are described and illustrated in relation to FIGS. 5 through 12. It will be understood by those of ordinary skill in the art that in addition to accepted screening instruments, the pre-therapeutic and therapeutic methods and systems of the present disclosure also contemplate using screening instruments for screening patients that are developed for a specific purpose.

Regarding the PROMIS screening tool, some beneficial features of PROMIS measures have greater precision than most conventional measures. Greater precision (less error) enhances power in a less costly way than increasing sample size. In one aspect, PROMIS measures have a larger range of measurement than most conventional measures, decreasing floor and ceiling effects as a result. Further, PROMIS measures do all this with fewer items than conventional measures, thereby decreasing respondent burden. When used as computer adaptive tests, PROMIS measures usually require 4-6 items for precise measurement of health-related constructs. PROMIS measures provide a common metric: the T-score (mean=50, standard deviation=10). In most cases 50 equals the mean in the U.S. general population. This metric has also been linked to many other conventional measures, and even if other measures are used, it may be possible to report results on the PROMIS metric, a considerable advantage for ensuring comparability across studies. In another aspect, PROMIS measures can be administered alongside Neuro-QoL™, ASCQ-Me®, and NIH Toolbox® measures that assess other aspects of health and function.

Regarding the PROMIS-57 screening tool, is a 57-question instrument covering seven domains-physical function, anxiety, depression, fatigue, pain, sleep disturbance, and social functioning.

Referring to FIGS. 5A-5C, the BDI, for example, includes 21 questions, and has an accepted interpretation in the medical oncology community. With a score of 1-10, for example, a patient may be categorized as “these ups and downs are considered normal.” With a score of 11-16 a patient may have “mild mood disturbance.” With a score of 17-20 a patient may have “borderline clinical depression,” a score of 21-30 may be categorized as “moderate depression,” 31-40 as “severe depression,” and over 40 as “extreme depression.”

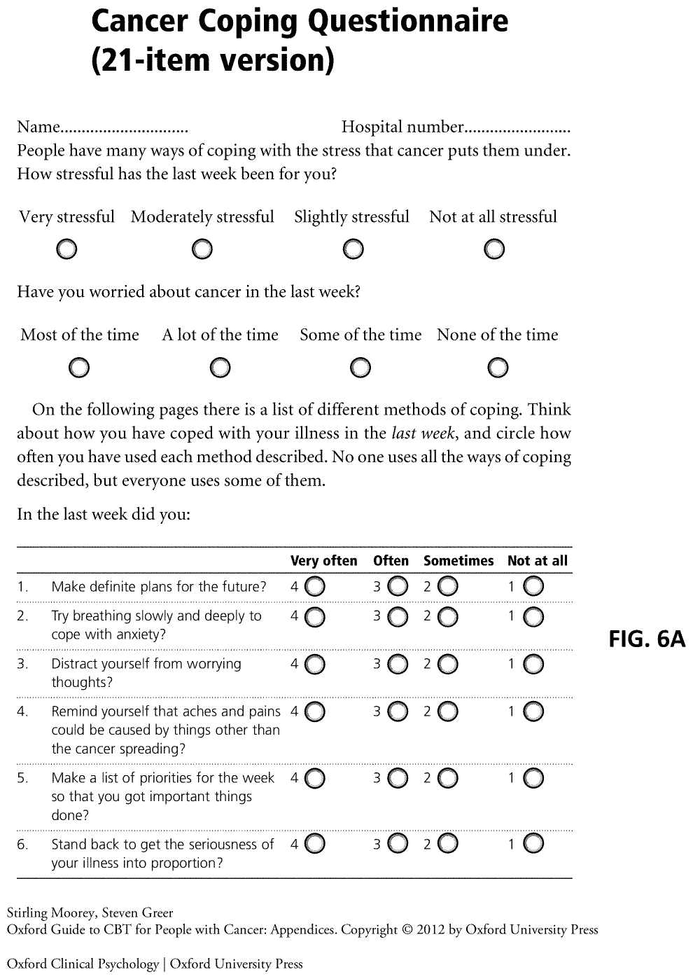

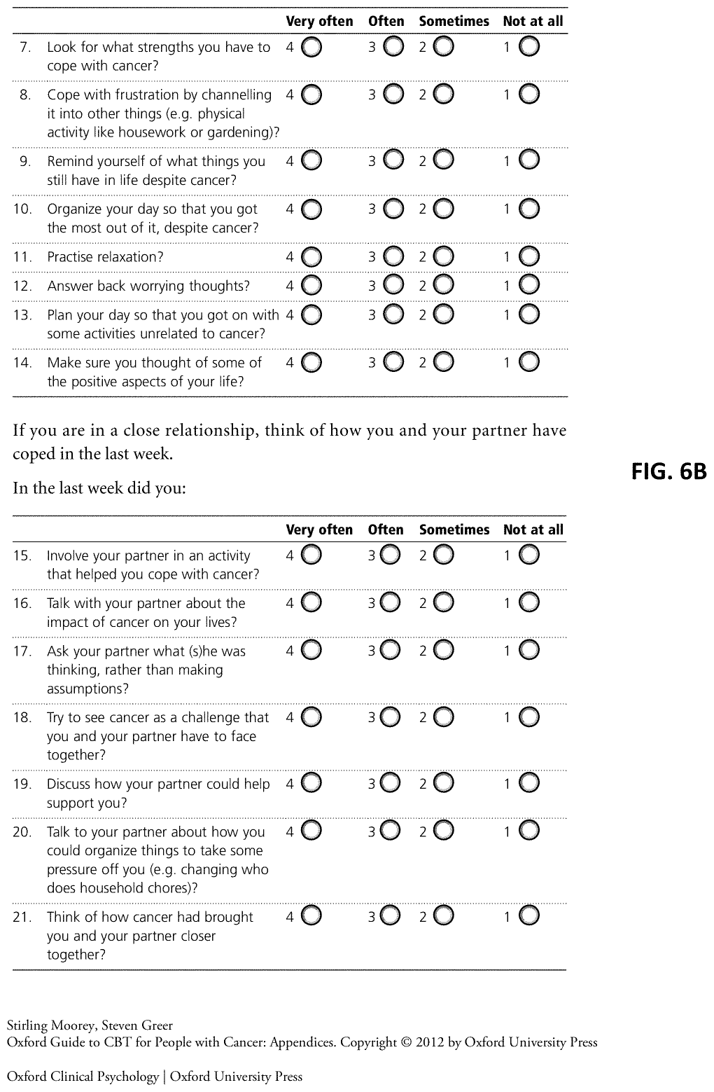

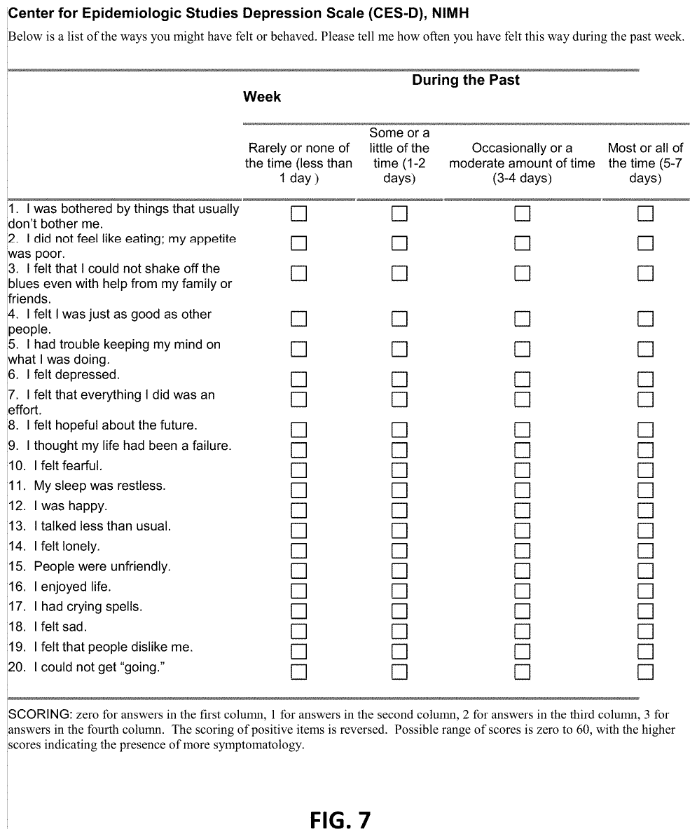

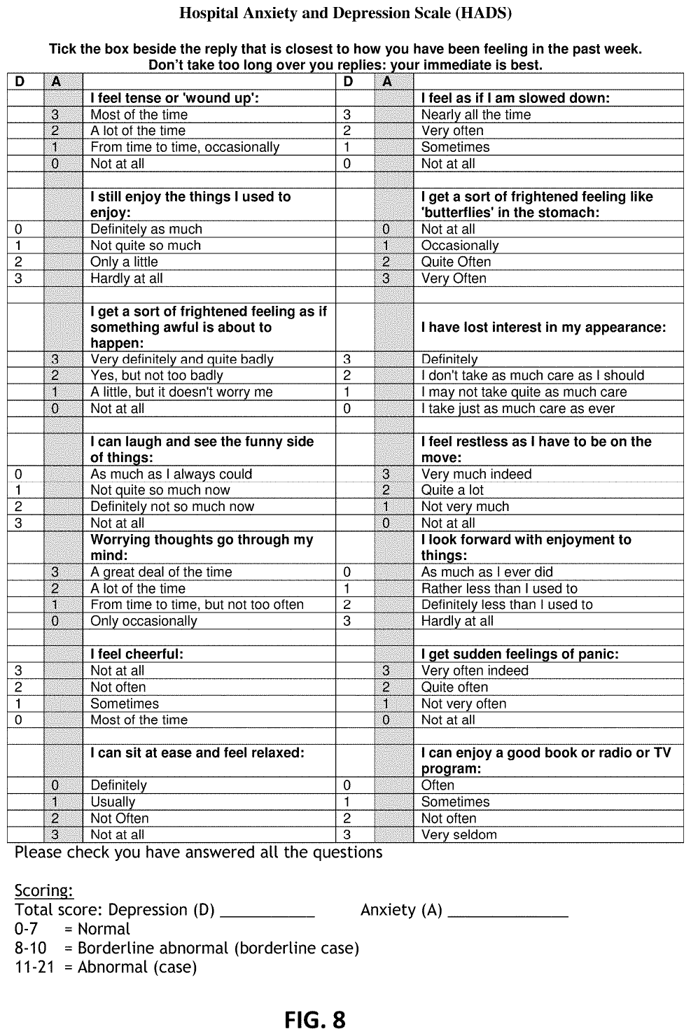

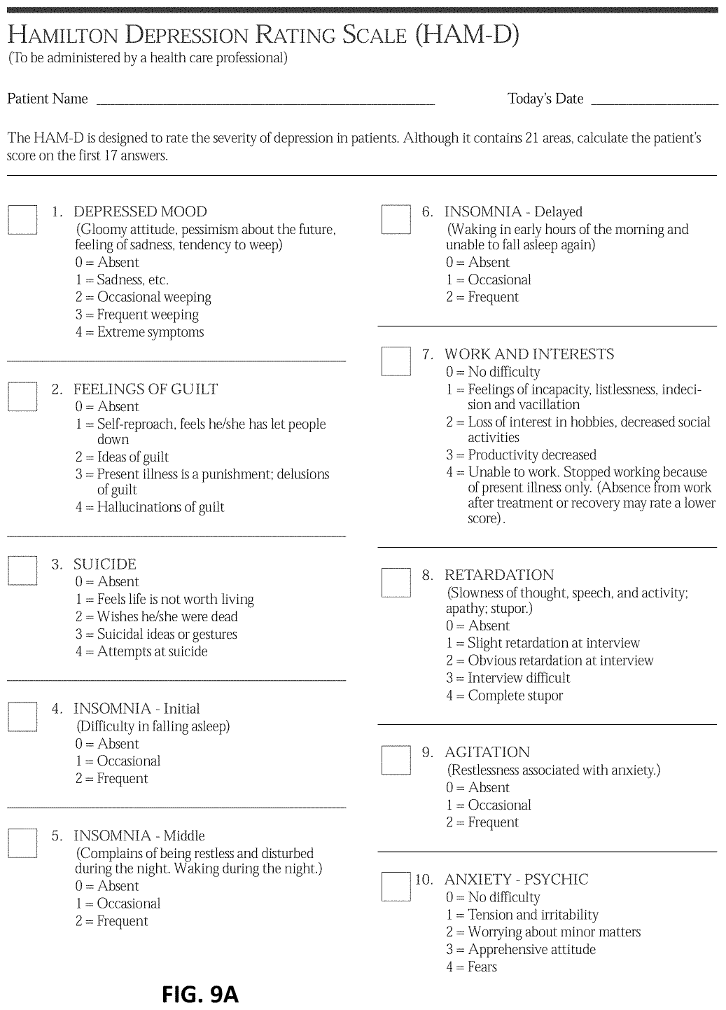

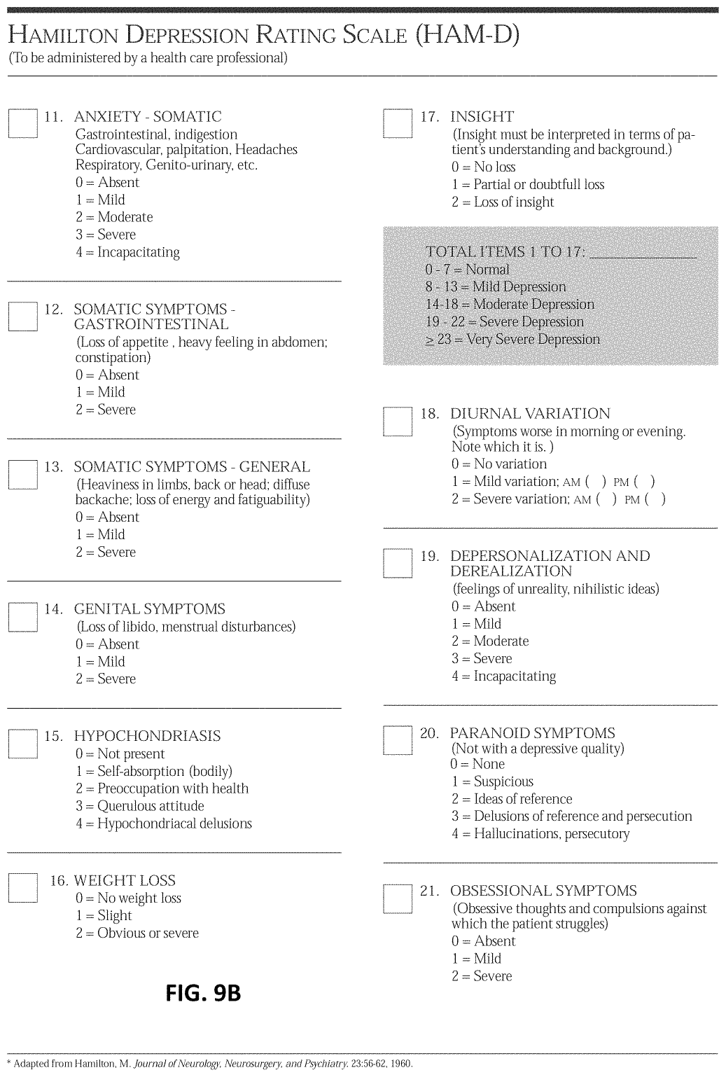

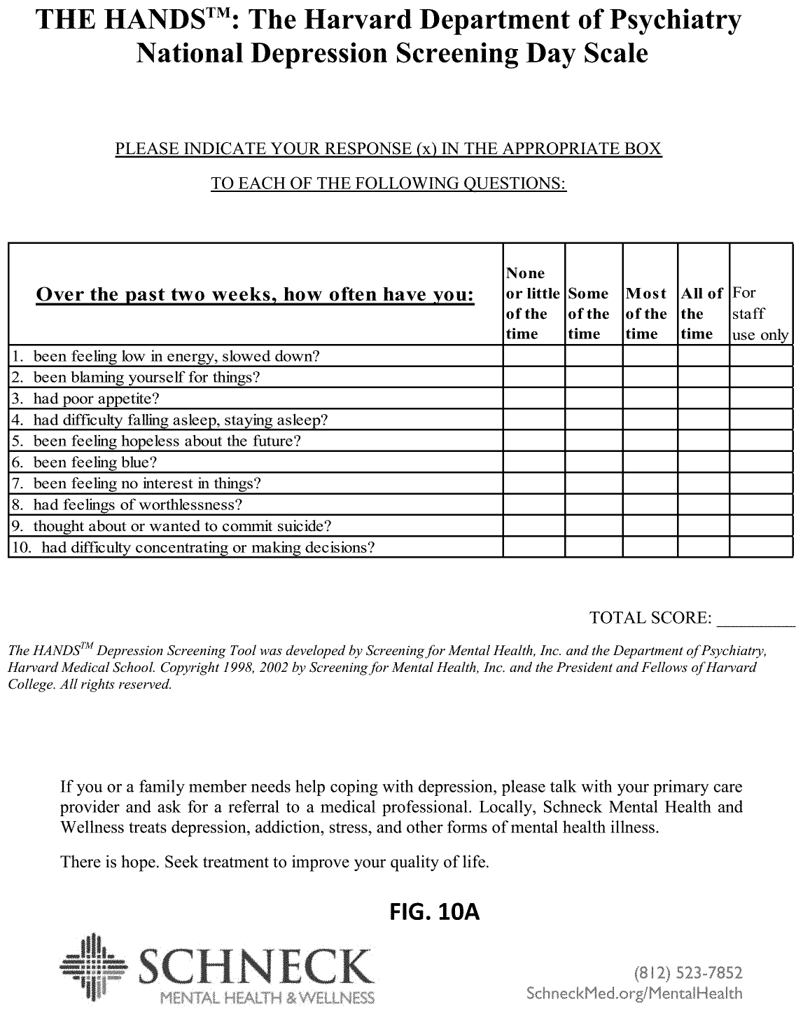

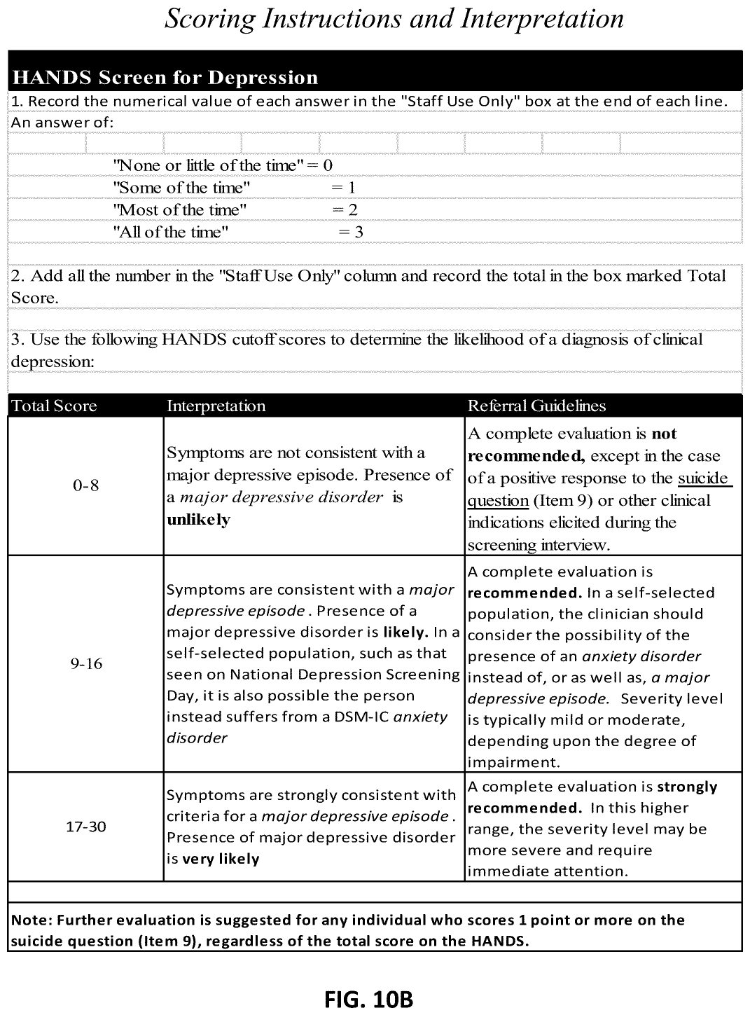

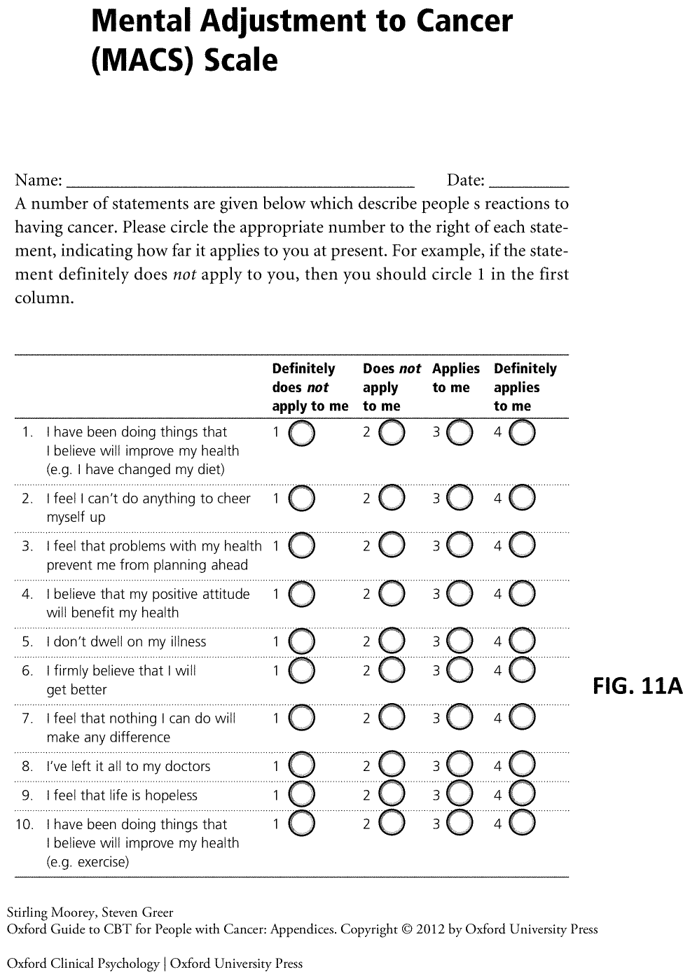

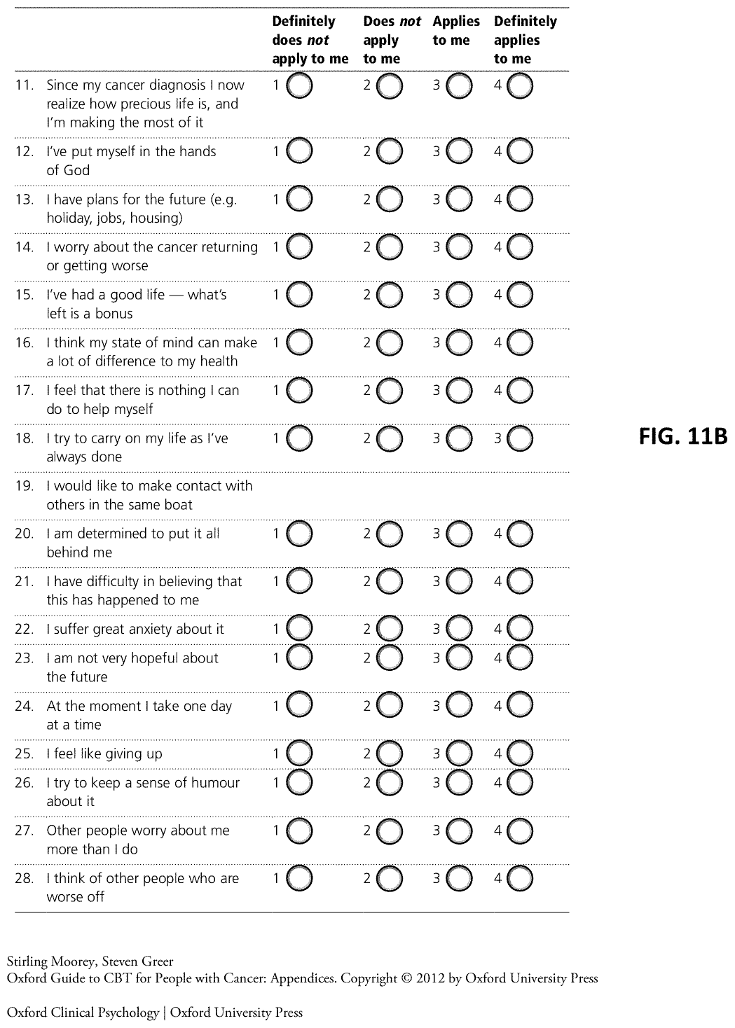

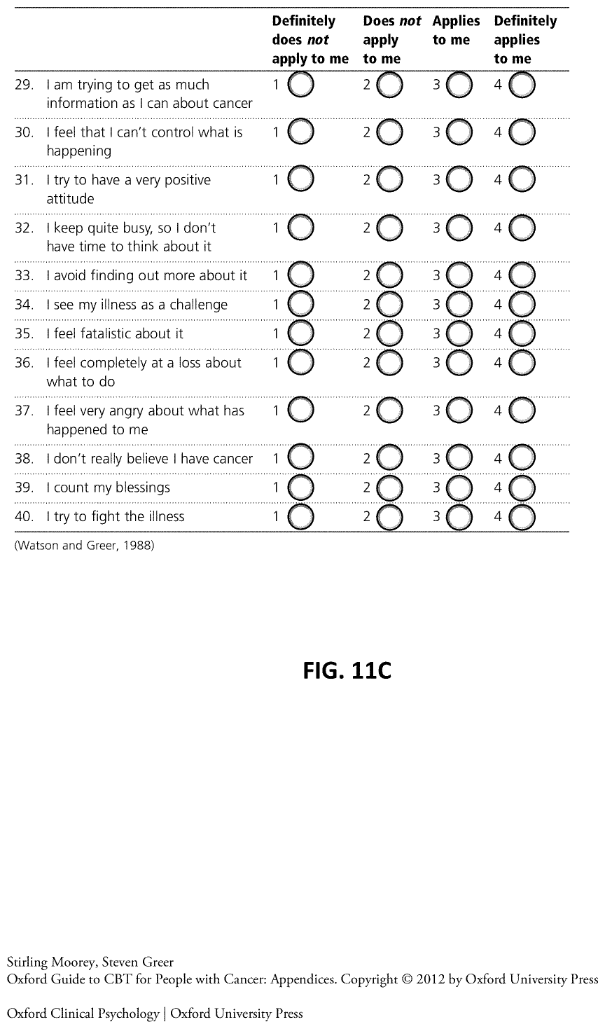

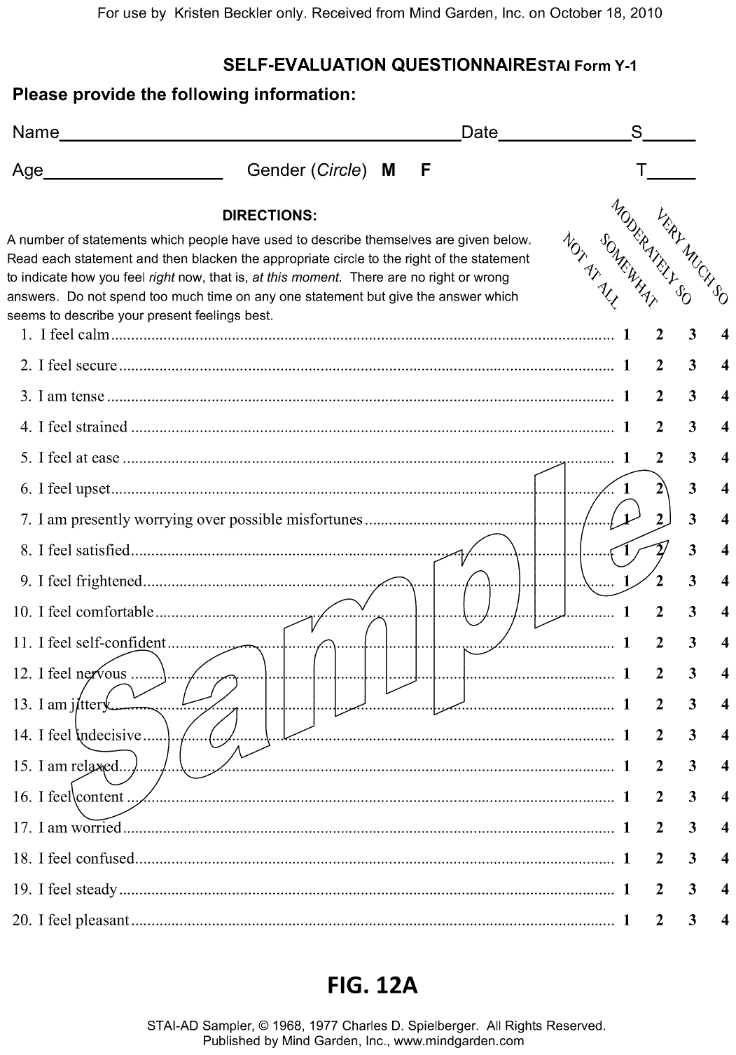

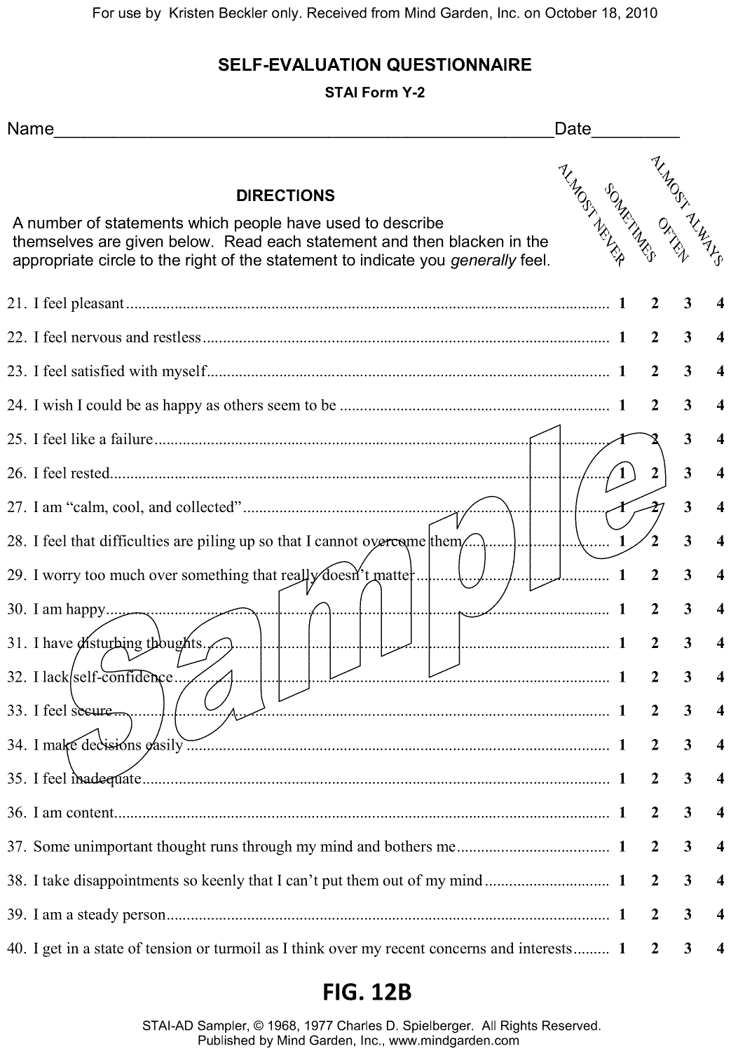

Referring to FIGS. 6A-6B, the CCQ, for example, includes 21 item, web-based psychosocial distress screen, referral and follow-up program. Referring to FIG. 7, the CES-D, for example, includes 20 questions. Referring to FIG. 8, the HADS, for example, includes 14 questions. Referring to FIGS. 9A-9B, the HAM-D, for example, includes 21 questions. Referring to FIGS. 10A-10B, the HANDS, for example, includes 21 questions. Referring to FIGS. 11A-11C, the MACS, for example, includes 40 questions. Referring to FIGS. 12A-12B, the MACS, for example, includes 40 questions. These example screening tools have an accepted interpretation in the medical community.

Referring back to FIGS. 1A-1B, at block 106, an interface between the patient and the system may be established. In some implementations, the interface may be a cloud Internet or 4-5G mobilebased, HIPAA-compliant protected account. In one aspect of the disclosure, a digital environment may be created for the patient, in which the patient may be able to establish a relationship with a particular healthcare avatar/therapist (interchangeably referred to herein as a digital or virtual image), virtual/live group of patients and group moderator, for example. The digital environment may also enable the patient to establish familiarity with the system and with the digital therapist. In accordance with one aspect, the digital therapist may guide the patient through the questionnaires, in lieu of just being handed a piece of paper on a checklist in the HCP waiting room, for example. The patient may be able to interact with, enter into a relationship with the digital therapist, and select a particular archetype of a digital therapist. For example, the patient may be presented with, e.g., five choices of different kinds of virtual healthcare providers (e.g., male/female, young/elderly, scientific/generalist), who may guide the patient through and administer the screening instruments. In addition, the digital therapist may be able to answer questions, or clarify questions, in a standardized way, which may be made consistent across all patients, for example. In accordance with one aspect, the example algorithm used, through implementation of artificial intelligence and self-learning (as described in more detail below), may acquire the ability to answer similar questions in a consistent way among different patients.

During patient diagnosis, blood tests may be run to determine the presence of any bio/chemical or epigenetic markers which may indicate a predisposition toward psycho-social comorbidities related to an oncology diagnosis, for example.

In accordance with one aspect, a virtual treatment room or virtual physician's office providing computer-generated interaction may be presented, viewable on a screen in the patient's home, e.g., a 60-inch flat screen, iPad, iPhone, or other device with comparable capabilities.

Alternatively, the patient may be able to use goggles or another interactive device at home or another location that would enable the patient to enter the virtual treatment room or virtual physician's office, for example.

At block 107 the system 100 may introduce, reinforce, model and instill adaptation skills to overcome adjustment or depressive disorders.

In accordance with one aspect, it may be important to understand how the scores evolve over time, e.g., at discrete regular intervals, and identify changes from the baseline score at block 108. Visual, written and oral cues of depression, anxiety and distress, among other conditions may be assessed using validated oncology care cues and emerging facial and voice-recognition technology, for example.

At block 110, using a pre-specified wellness change or otherwise based on the identified changes from the baseline score, for example, a determination may be made as to whether the communication initiation threshold is met.

An HCP using the pre-therapeutic system in accordance with aspects of the present disclosure may establish various thresholds, e.g., establishing that an increase of more than 10 on the BDI is a special situation that needs to be algorithmically flagged. The information may be sent to the HCP via any electronic means available, for example, e-mail, text, or integration with the pre-therapeutic and therapeutic system, in accordance with aspects of the present disclosure.

As another example, the HCP may determine that any variation in the screening score should be sustained for a minimum of several days before notifying the patient that there may be information and data that should be shared with the patient's healthcare team. Alternatively, for example, an HCP may decide that several consecutive spikes may warrant alerting the healthcare team. As will be recognized by those skilled in the art, various other thresholds may also be established by the HCP.

In accordance with one aspect, an HCP may also customize the system. For example, an HCP may be desirous of collecting data based on a scoring system that the HCP is familiar with and has used historically in their practice. In addition, the HCP may reduce the number of screening instruments that a patient interacts with, for example. Conversely, the HCP may decide that it is beneficial for the patient to develop scoring against more than one screening instrument. It will be recognized by those skilled in the art that various types of customization may be implemented by the HCP. Patient outcomes may be compared to their own baseline measurements, to measurements of other patients undergoing therapy, or historical data found in medical literature, for example. In accordance with one aspect, the pre-therapeutic and therapeutic methods and systems may have underlying artificial intelligence or machine learning that collects and uses user data to improve their own capabilities.

In accordance with one aspect, creating longitudinal data or repeating the assessment over time may enable an HCP to witness increases or decreases in depression and related symptoms over a meaningful interval.

In accordance with one aspect, the system may provide information to the patient regarding departures from the baseline. A block 112, the patient may elect to participate in the assessment, based on the information provided by the system, among other factors. Upon receiving the patient's election, the information may be aggregated and provided to the patient's healthcare team, enabling the HCP, e.g., a nurse, physician or social worker, to contact the patient and discuss next steps, such as a therapeutic choice, for example.

In accordance with one aspect of the disclosure, all information may be packaged and provided to the HCP in a pre-specified format, at block 114, so as to eliminate uncertainty in the decision of the patient to contact the HCP. That is, uncertainty regarding the patient being able to express what the patient is experiencing, when to contact the HCP, who exactly to contact at the HCP office, etc., may be reduced or eliminated, as all suitable information may be transmitted to the HCP in a standardized manner, while the patient may remain in control of the decision whether to proceed with the assessment at block 112. In accordance with one variation, the patient may their decision to proceed with the assessment at any time.

At block 116 (FIG. 1B), the information may be extracted, packaged and transmitted to the HCP. The HCP may be provided with an alternative interface (e.g., cloud-based) to the system, so as to receive and transmit data. At block 116, the data may leave the patient's secure, cloud-based file, and with the patient's authorization may be transmitted to the HCP or Electronic Health Record (EHR) workflow software. For example, An Electronic Health Record (EHR) is an electronic version of a patients medical history, that is maintained by the provider over time, and may include all of the key administrative clinical data relevant to that persons care under a particular provider, including demographics, progress notes, problems, medications, vital signs, past medical history, immunizations, laboratory data and radiology reports The EHR automates access to information and has the potential to streamline the clinician's workflow. The EHR also has the ability to support other care-related activities directly or indirectly through various interfaces, including evidence-based decision support, quality management, and outcomes reporting.

For example, the system may include an artificial intelligent (AI) algorithm (e.g., a learning algorithm), which may execute a smooth handoff of the patient/user to a human, in situations where such a handoff becomes necessary. In accordance with one aspect, the system may enable the patient to take risk-mitigating steps, in the case of a risk of suicide, for example. That is, if the term “suicide” is mentioned, for example, there may be a specific protocol in place for taking action. Specific triggers, e.g., event-based, term-based, or queue-based, may be implemented for serious situations, such as the risk of suicide, for example. In some implementations, if the patient is impaired and incapable of clearly communicating the circumstances necessitating seeking assistance, the system may be able to route acute patients to the emergency room and provide explanations to a specialist, as to the essence of such circumstances. The communicated circumstances may include the symptoms the patient is experiencing and the patient's longitudinal data, for example, among other data, thereby assisting and/or obviating the necessity of the patient explaining what the patient is experiencing to the human HCP. In addition, the circumstances may include data relating to labs, vital signs, bloodwork, enzyme testing, or electrocardiogram (EKG) information, among other data, which may be obtained via the system or via a third-party provider, for example.

In accordance with some aspects, that system algorithm may initiate patient interaction and other activity on a basic level, without having a specific downstream solution for every situation, whether it be relationship-related, cognitive, or behavioral, for example, and may only be able to reach a specific point in providing responses/solutions. That is, it is envisioned that the algorithm may initially have limitations in terms of problem-solving capabilities. In accordance with one aspect, for example, the algorithm may determine that there is no further logical step available, and may require handoff of the patient to a human HCP. Nevertheless, with use and experience, including using the learning capabilities of AI, for example, the digital therapist algorithm may become more complete and able to be prepared for likely future conversations/problem solving based on the direction of past conversations, previous problem-solving, and other interactions with patients.

Once the data is transmitted to the HCP, an evaluation may be made by the HCP of the patient's wellness score on the basis of the transmitted data. This evaluation may be provided to the patient and/or the system at block 118. Many HCP group practices and comprehensive care facilities today have enhanced services, frequently including a dedicated psychologist or oncology social worker who may provide a referral to a psychologist, for example. In some instances, e.g., during survivorship, and in some cases during treatment, the oncology social worker may also leverage a referral to the patient's general practitioner or primary care physician. The attending physician/HCP may then make a particular therapeutic decision/clinical judgement, which may be transmitted to the patient and/or the system at block 120.

In accordance with some implementations, if a decision is made not to treat, the system may revert to block 118, to determine, e.g., if any additional data is necessary, and how to respond to the patient, among other determinations.

If a decision to treat is made, at block 122, a communication link may be established between the HCP and the patient to discuss the urgency of treatment, therapeutic options, and execution of the clinical decision, among other factors. In addition, a communication link between the HCP, the patient, the office staff management may be established, and medical claims may be processed and submitted. The communication links may be established, e.g., via electronic means as determined by the HCP or practice-specific operations, for example.

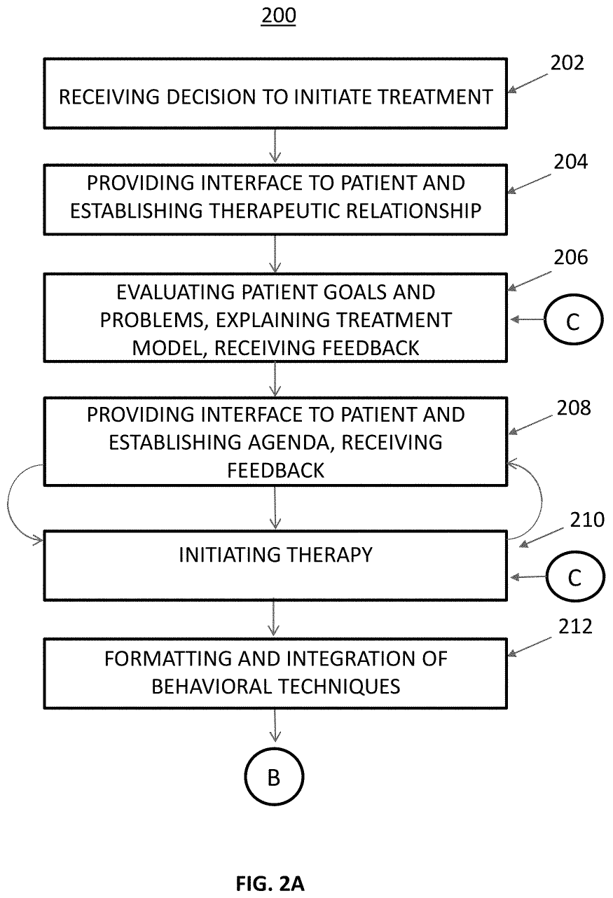

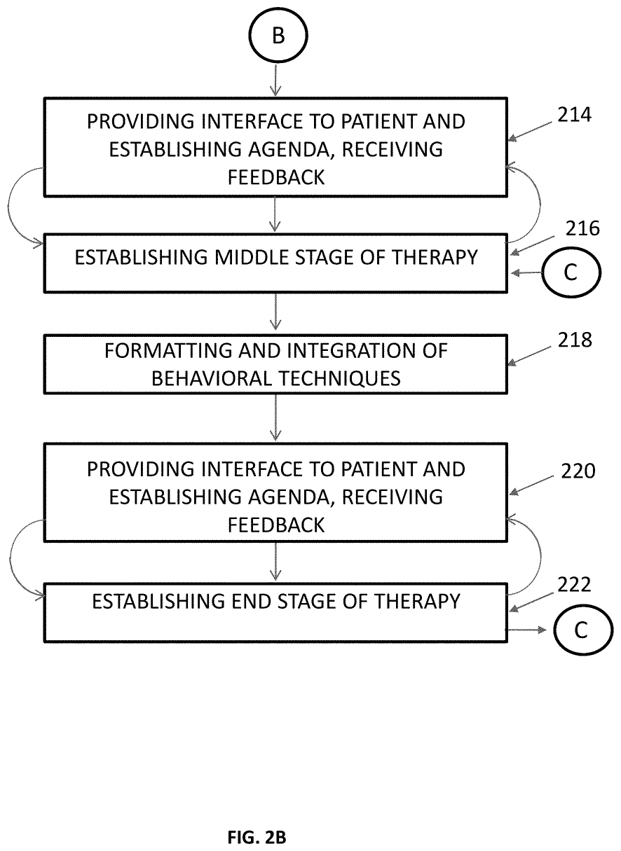

Referring now to FIGS. 2A-2B, therein shown is a flowchart of an example method for therapeutic processing and treatment, according to an aspect of the disclosure. Based on the method described in FIGS. 1A-1B, for example, a patient may make the decision to initiate treatment and may communicate that decision to the system at block 202. For example, the patient may communicate the decision by logging into the system and completing certain authorizations via a patient interface to begin the treatment program for one or more signs of depression and/or psychosocial comorbidities, including anxiety, depression, fatigue, insomnia, pain, or cognitive disruption, for example.

In accordance with some aspects, a decision may be made regarding the schedule and duration of treatment based on pre-determined thresholds in the system (e.g., duration of treatment is generally recommended to be between 8 and 12 weeks), input from the HCP, and/or the patient's responses to certain questions. The patient and the HCP may interact with the system via their respective interfaces to provide their input.

Other factors may also influence the schedule and duration of treatment, such as the specific diagnosis, e.g., anxiety, depression, insomnia, etc., the severity of the condition (there are accepted protocols for each type of comorbidity in the medical oncology community), and whether the patient is responding well to treatment after the first few sessions with the digital device, among other factors. Each of the relevant factors may be weighted respectively prior to making a decision regarding schedule and duration of treatment. One option, if the patient is feeling well, for example, may be to return to the longitudinal monitoring at block 108 of FIG. 1A. In general, the goal is for the patient to return to the baseline psychosocial wellness score established at block 104 of FIG. 1A, and/or to improve from that baseline.

At block 204, an interface to the system may be provided to the patient, and a therapeutic relationship may be established between the patient and the system, e.g., with a digital healthcare avatar/therapist, along the lines discussed above in relation to FIGS. 1A-B. With respect to cancer patients in particular, a credible therapeutic relationship may be important. That is, in terms of actions and interactions, the system may need to operate at a sufficient speed, for example, where the patient and the digital therapist can have a two-way conversation that is akin to a logical and humanlike interaction in a photorealistic environment. One advantage, in accordance with aspects of the present disclosure, is that the digital healthcare avatar/therapist may have faster access to more resources (e.g., a vast array of database information), which may be unavailable to, or not easily accessible or assimilatable by, a human therapist, and may be able to respond to a patient's questions faster, and/or with more in-depth responses and/or with responses that are more consistently aligned with evolving treatment practice. In addition, receiving immediate responses may be more satisfying to the patient, as compared to waiting for a human to conduct research and providing a response at a later time, for example.

In accordance with some aspects, a measure of a successful relationship between a patient and the system may be based on patient feedback, for example.

As described in more detail below, the system allows for multiple opportunities for patients to provide feedback, e.g., at blocks 206, 208, 214, and 220, for example.

At block 206, an evaluation may be performed of the patient's goals and problems, and an explanation of the treatment model may be provided. These functions may include gleaning an understanding of the problems the patient is facing, the patient's goals, and how each should be prioritized. A therapeutic structure may be determined. For example, the regularity, number, and duration of sessions may be decided, along with specific assignments, so that the patient and the system are able to set expectations. In accordance with some aspects, an advantage of the digital therapist may be the ability of the system to readily pre-specify and handle calendaring and scheduling for the patient. In accordance with another aspect, the treatment model may also take into account experiences from prior patients. In addition the treatment model may provide the patients with hints/suggestions from experienced patients.

In some implementations, the system may provide an activity template (e.g., eight sessions of 30 minutes each may be determined as necessary over one month), but also enable the patient to customize the template to the patient's own calendar and needs. In addition, the system may provide homework assignments to the patient, some of which may be graded and tracked digitally. At the conclusion of the session, the patient may be provided an opportunity to generate feedback to the system, e.g., regarding whether interaction with the system is useful and/or enjoyable to the patient.

In accordance with some aspects, the system may provide a sleep log template. For example, the patient may self-report to the system dates, times, duration, and quality of the sleep. In accordance with another aspect of the system, the system may provide a stress level log. For example, the patient may self-report in the system the stress level of a patient throughout their day, situations that triggered the stress, along with dates, times and events.

At block 208, an interface to the system may be provided to the patient, and an agenda may be established. Agenda setting may be important to ensure that a patient's current needs are informing the direction of each session, rather than strictly following an a priori structure that was set in the initial session at block 206, for example. A process of guided discovery/collaborative empiricism may be implemented to deconstruct, test, and question a patient's beliefs, for example. In one aspect, the patient may collaborate, interact and/or participate with a group. For example, the group may be other current and/or former patients or the AI software. Cognitive behavioral therapy (CBT) may be used in some variations to encourage patients to pursue behaviors, activities, and/or other pursuits that are conducive to wellness. In accordance with some implementations, the AI and learning aspects of the system may be implemented via decision tree algorithms or Monte Carlo simulation, for example. In addition, specific triggers, e.g., event-based, term-based, or queue-based, may be implemented. Again the patient may be allowed an opportunity to provide feedback to the system, e.g., regarding whether interaction with the system is useful and/or enjoyable to the patient. Based on the patient feedback, the system may improve, learn, and adapt, as will be understood by those skilled in the art, in accordance with the principles of AI and/or evolving science in psychotherapy. The system for example, would encourage the patient to participate and interact with the system thereby also providing a group dynamic.

Frequent summarization of data may also prove important with respect to treatment. In accordance with one aspect, the ability of the system to access data may be advantageous in providing the ability to summarize progress made during the session. Progress in deconstructing complexity around ways of thinking or observations that a patient may have about the patient's life, condition, etc., and summarizing such progress may be validated to the patient. Otherwise, further assessment and clarification may be needed. In accordance with aspects of the present disclosure, handoff to a human caregiver, with some background information, may be provided at any suitable point in the process.

Among other advantages, the system in accordance with aspects of the present disclosure may be much less resource-intensive than required using only human HCPs at oncology clinics, for example, which currently may not have sufficient in-clinic resources for monitoring, e.g., forty or sixty patients every day.

At block 210, therapy may be initiated, including identification, assessment, and treatment of primary symptoms, for example. Another component may include behavioral treatment, e.g., encouraging a return to living an ordinary life. Some example of behavioral treatment may be Cognitive Behavior Therapy (CBT), Relaxation Therapy (RT), Problem-Solving Therapy (PST), Acceptance and Commitment Therapy (ACT), Cognitive Bias Modification (CBM), AIM, and Meta-cognitive Therapy (MCT). Embracing a cognitive model, collaborative empiricism, and encouraging open expression of feelings may be additional components of carrying out such therapy.

At block 212, formatting and integration of behavioral techniques may be provided. One component may be initiating, scheduling, booking relaxation training, including internal and external to the patient and in-home and out-of-home training, for example. These techniques may be fully coordinated and calendared with the patient's schedule via Google Assistant made by Google LLC of Mountain View, Calif., for example, among other software tools. As another component, activities may be scheduled, and homework may be graded. Future planning, including the ability to plan ahead for trips and other events to look forward to in the future, and behavioral experiments may be another component.

At block 214 (FIG. 2B), an interface to the system may be provided to the patient, and an agenda may be established. Agenda setting may be important to ensure that a patient's current needs are informing the direction of each session, rather than strictly following the a priori structure that was set in the initial session at block 206, for example. A process of guided discovery/collaborative empiricism may be implemented to deconstruct, treat, and/or question a patent's beliefs (e.g., negative beliefs). Cognitive behavioral therapy (CBT) may be used in some variations, such as to encourage patients to pursue behaviors, activities, and/or other pursuits that are conducive to wellness. In accordance with some implementations, the AI and learning aspects of the system may be implemented via decision tree algorithms or Monte Carlo simulation, for example. In addition, specific triggers, e.g., event-based, term-based, or queue-based, may be implemented. Again the patient may be provided with an opportunity to provide feedback to the system, e.g., regarding whether interaction with the system is useful and/or enjoyable to the patient. Based on the patient feedback, the system may improve, learn, and adapt, as will be understood by those skilled in the art, in accordance with the principles of AI.

At block 216, the middle stage of therapy may be established, including components such as thought monitoring and coping, and beginning to track dysfunctional thought. In accordance with one aspect, an example system in accordance with aspects of the present disclosure may track and record these items at the time they are occurring, without having to wait for an appointment with a healthcare provider, for example. Collecting input may be achieved via, e.g., mobile device interface and/or SMS messaging, among other devices and/or methods, for example voice via a microphone, video via a camera, etc. Patients may have the ability to personalize their problem solving processes using their own language and sequencing, for example. Another component may include establishing what is different and positive as a result of cancer, for example.

At block 220, formatting and integration of behavioral techniques may be provided. One component may include initiating, scheduling, booking relation training, including internal and external to the patient, and in-home and out-of-home training, for example. These techniques may be fully coordinated and calendared with the patient's schedule via Google Assistant, for example, among other software tools. As another component, activities may be scheduled, and homework may be graded. Future planning, including the ability to plan ahead for trips and other events to look forward to in the future, and behavioral experiments may be yet another component.

At block 222, the end stage of therapy may be established, which may include a focus on preventing relapse into depression or anxiety, for example. Planning for future and long-term goals, may be another component of the end stage of therapy, and may be an important part of moving out of a period of crisis and into a period of reflective processing, as well as identifying and recording any underlying assumptions. Addressing survivorship and planning for a post-cancer lifestyle may be yet a further component, including a focus on the behavioral techniques that should be included in the patient's daily life going forward after therapy, for example. At the completion of therapy, a summary of the therapy may be provided for discussion with a physician. Returning to the pre-therapeutic functionality, discussed in more detail above with respect to FIGS. 1A-B, may also be contemplated, allowing for the patient's self-reported data to be returned and compared with both the baseline and the prior longitudinal data gathered at blocks 104 and 108.

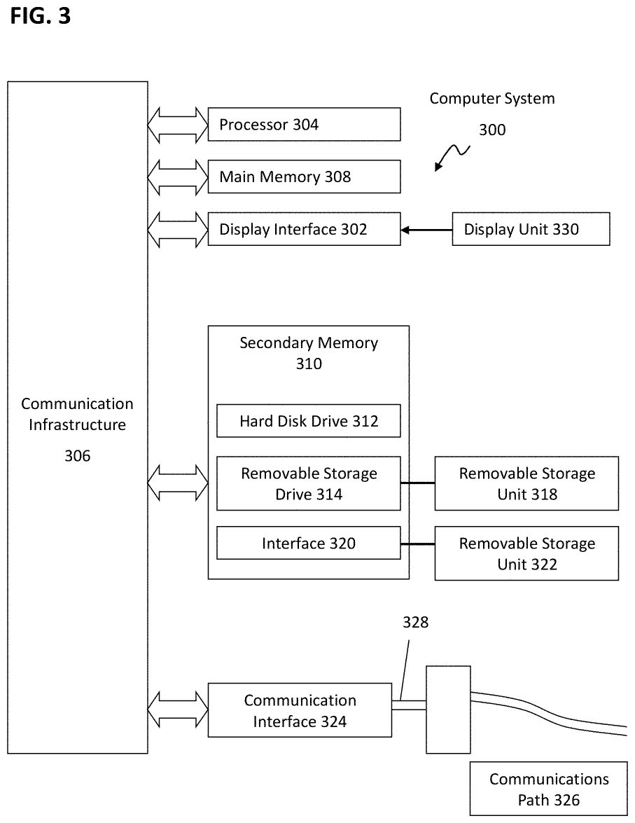

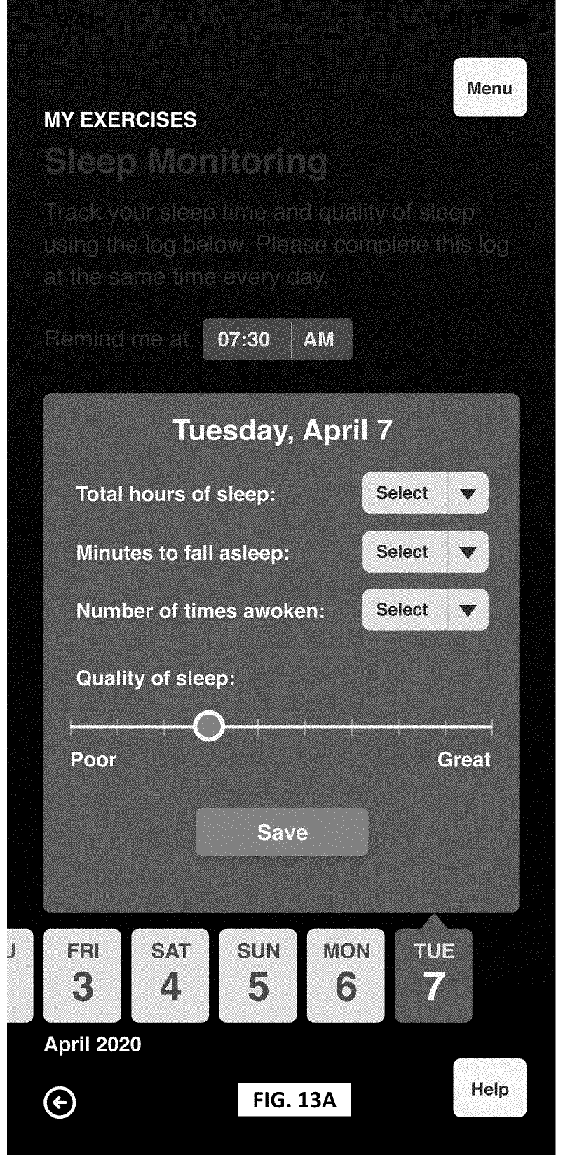

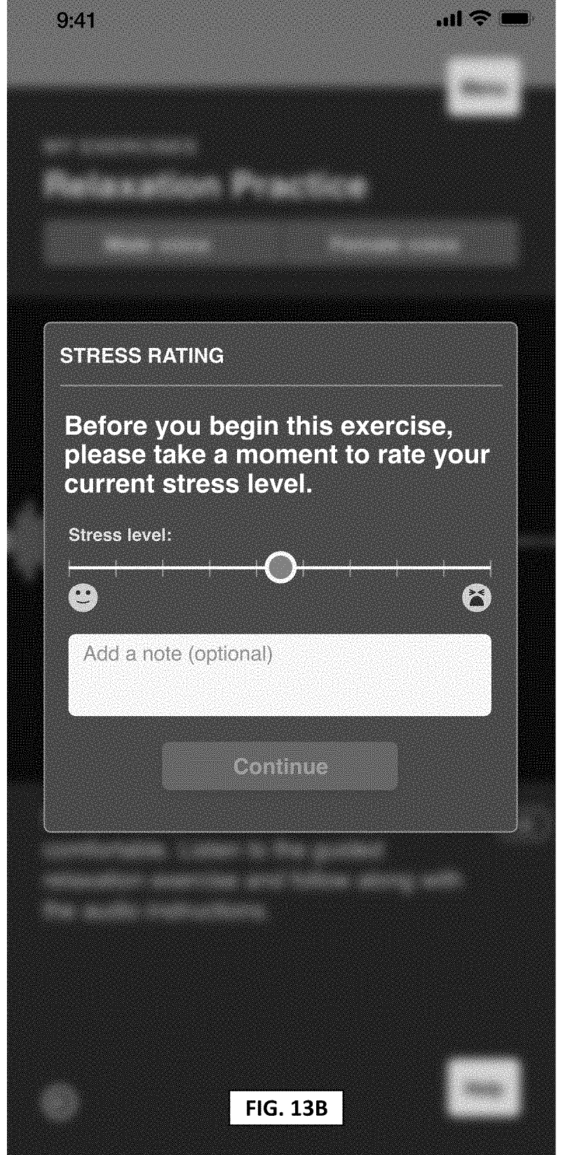

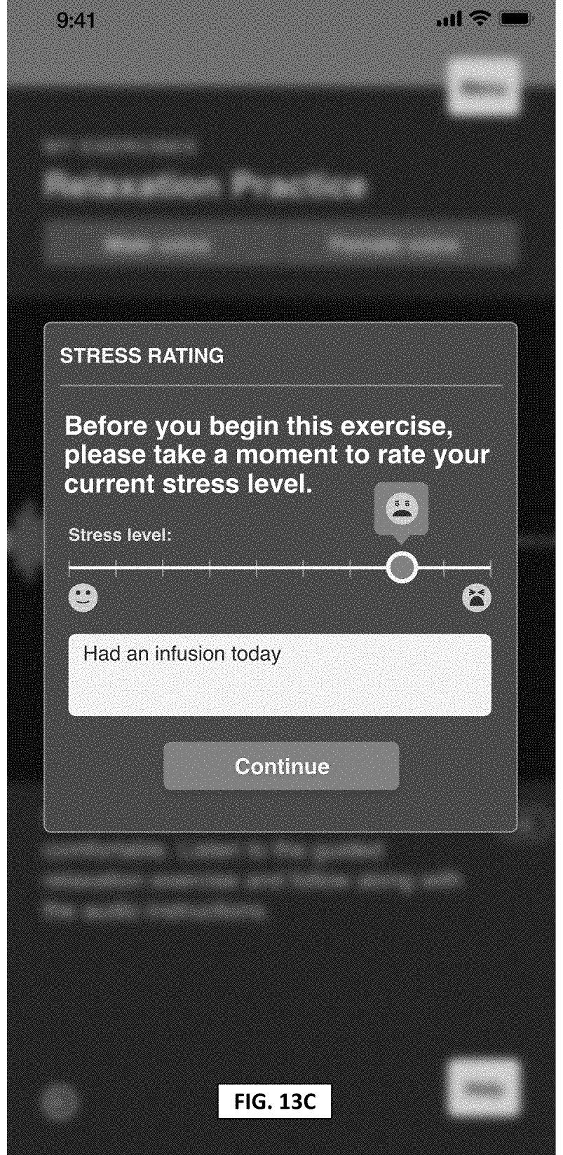

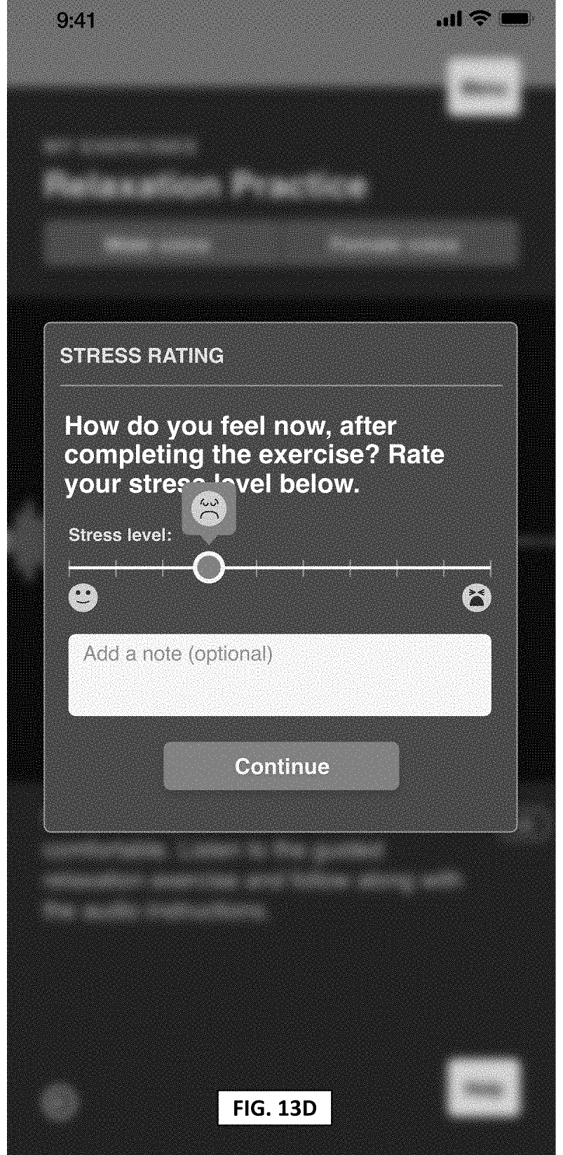

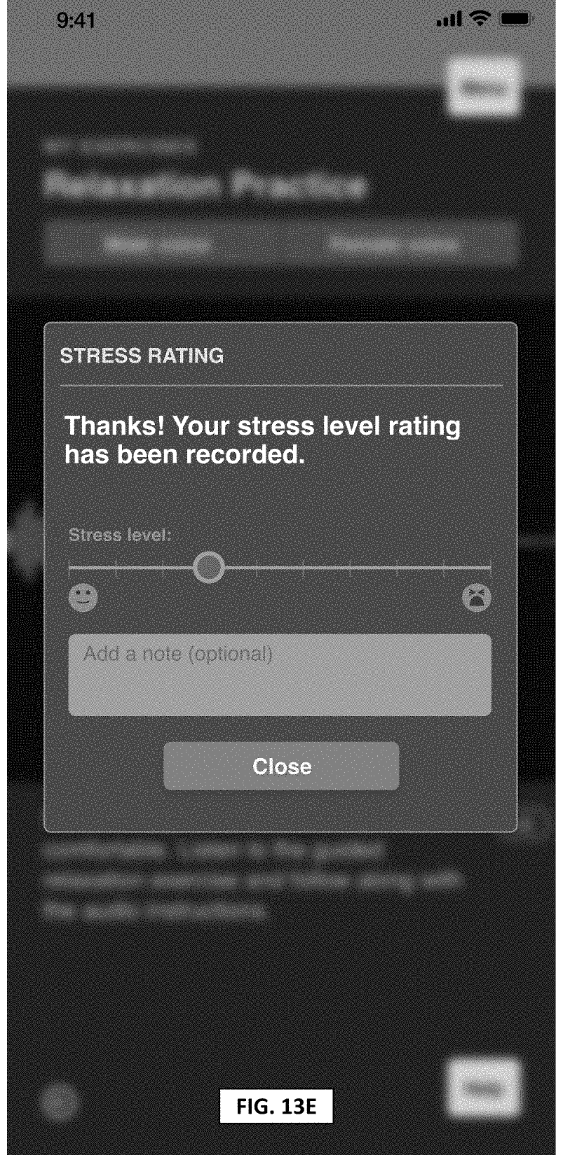

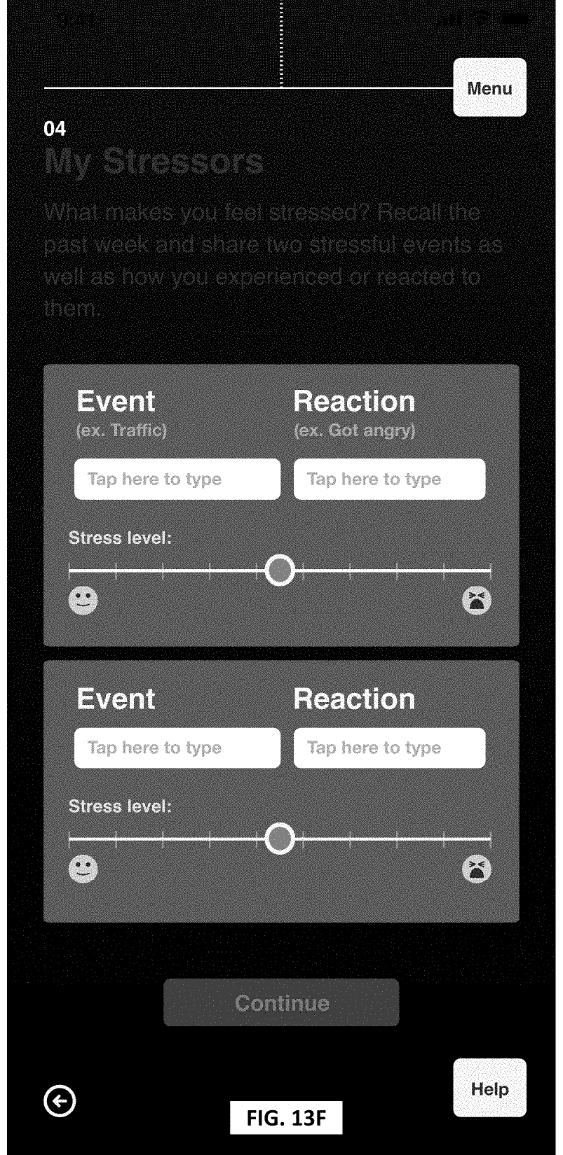

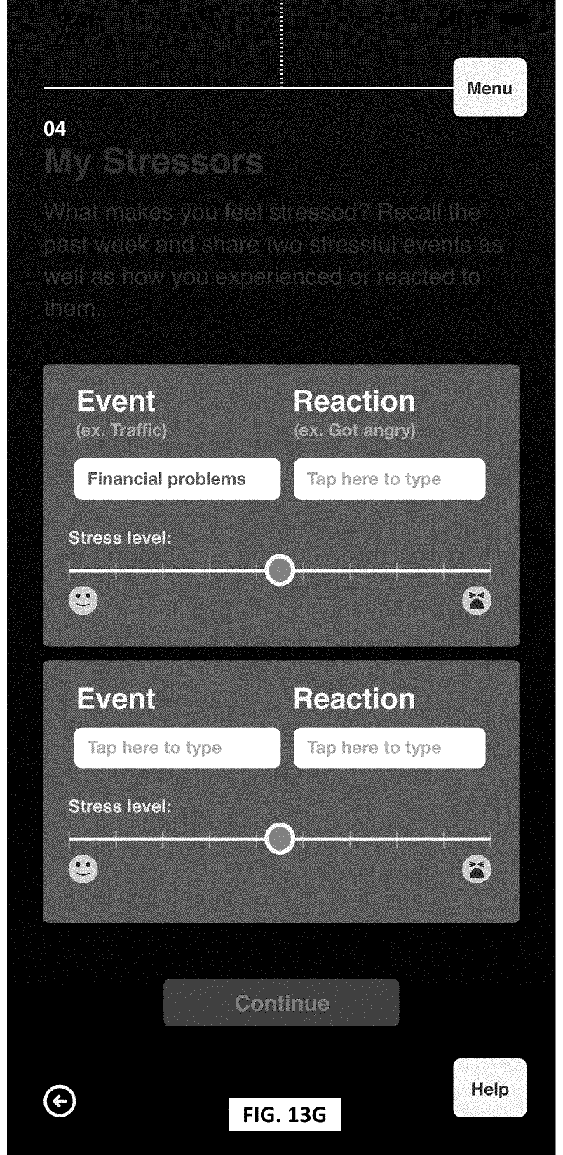

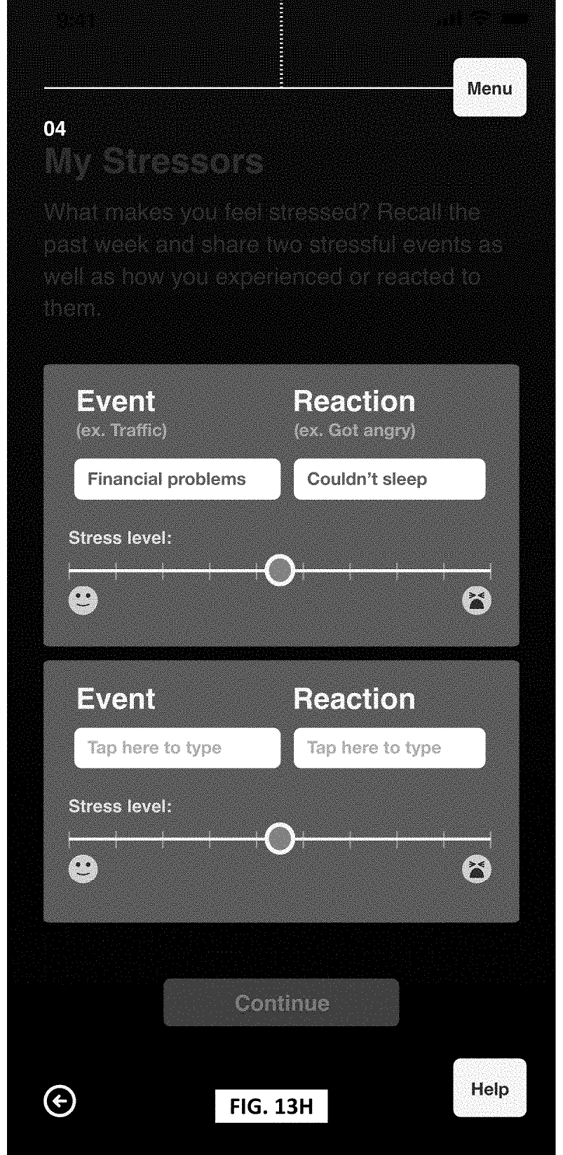

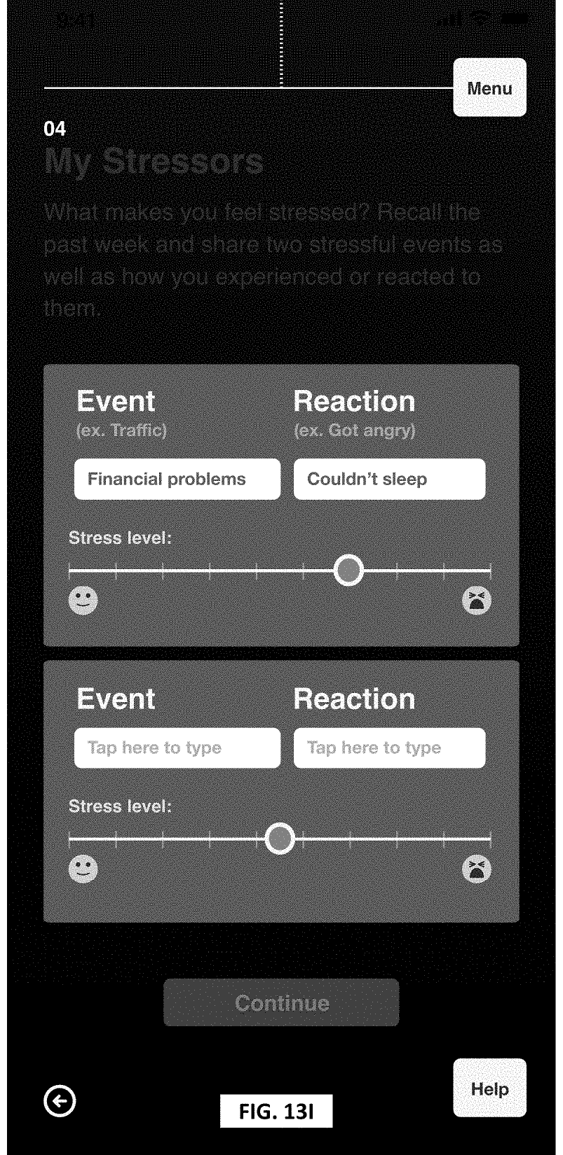

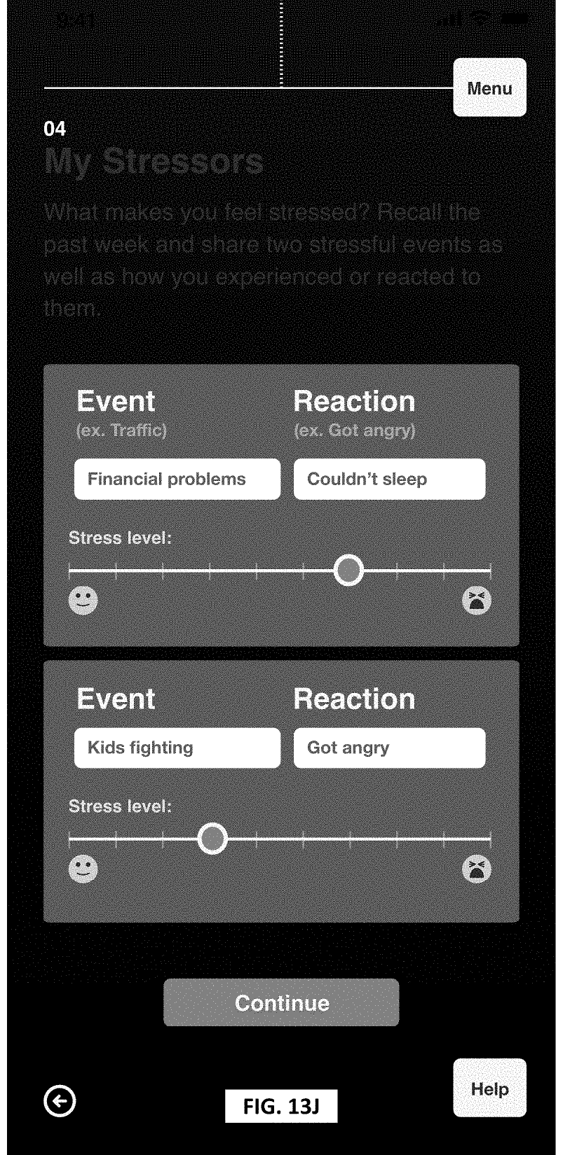

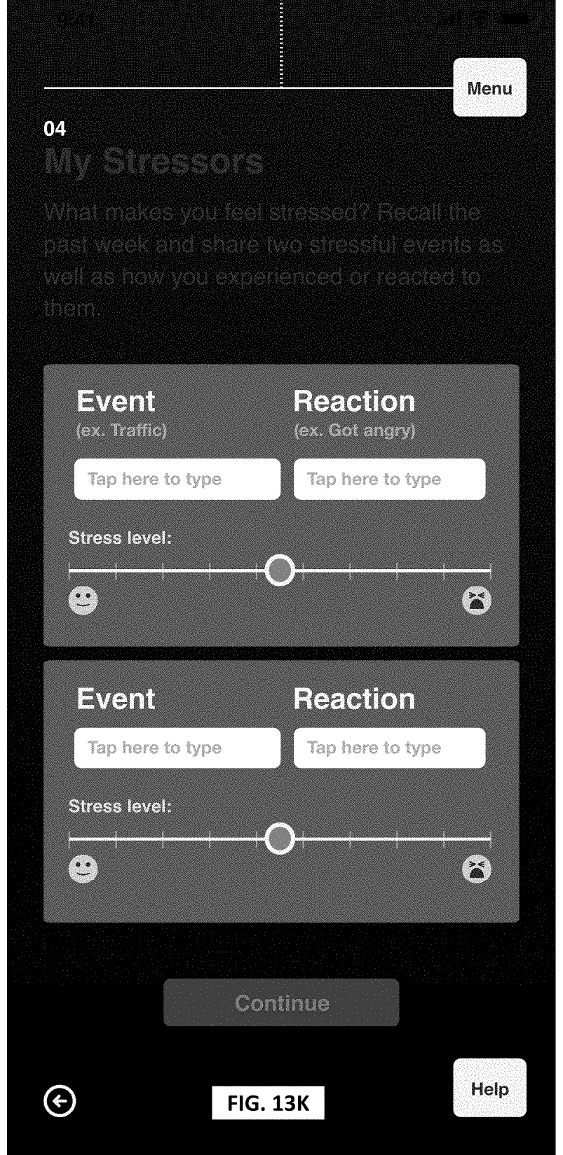

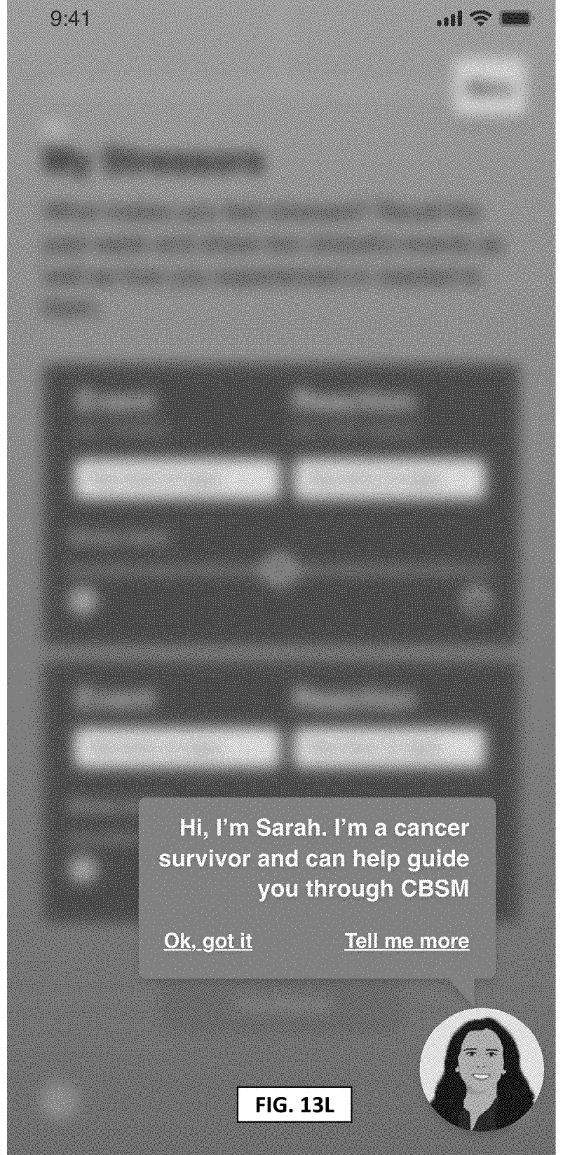

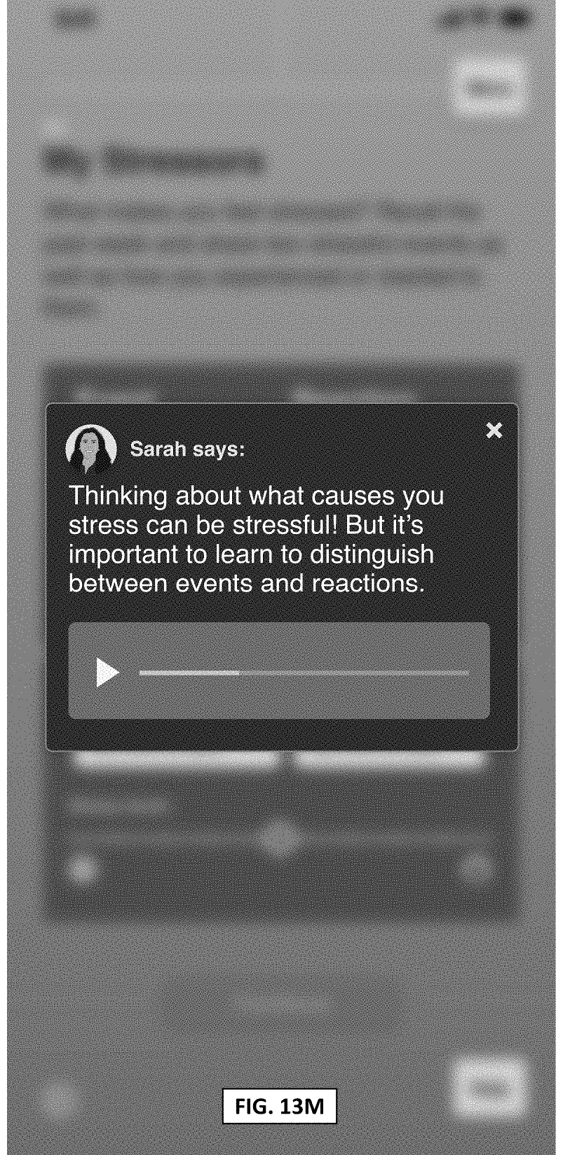

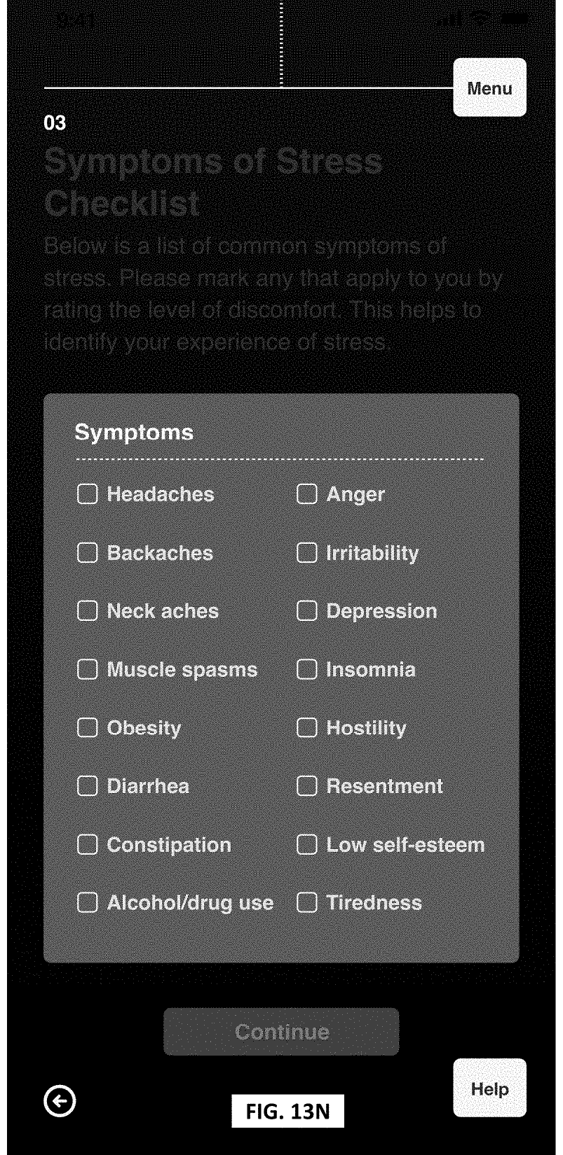

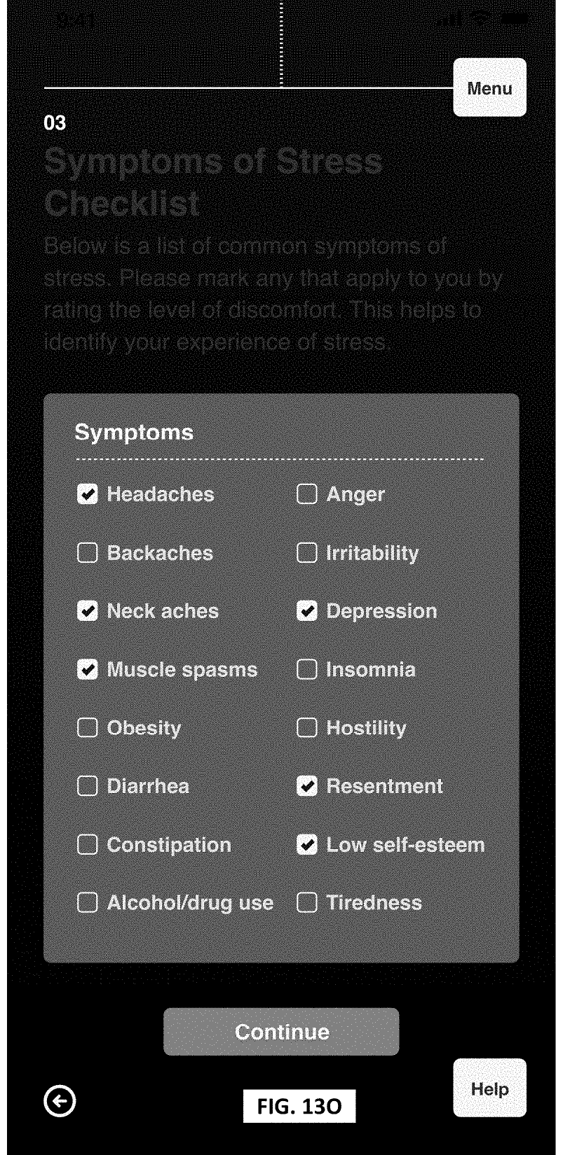

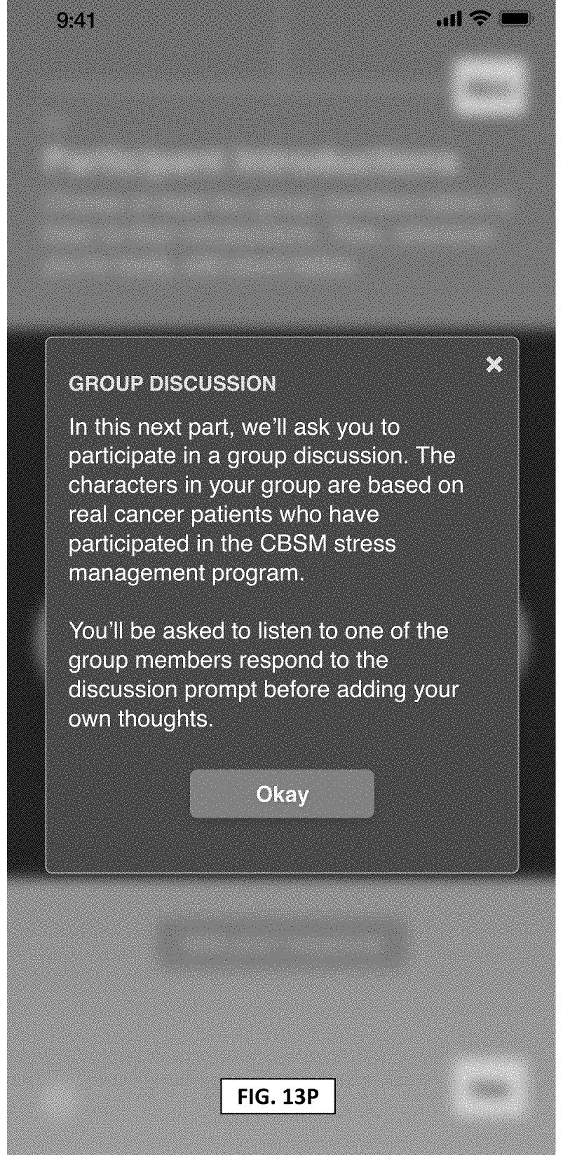

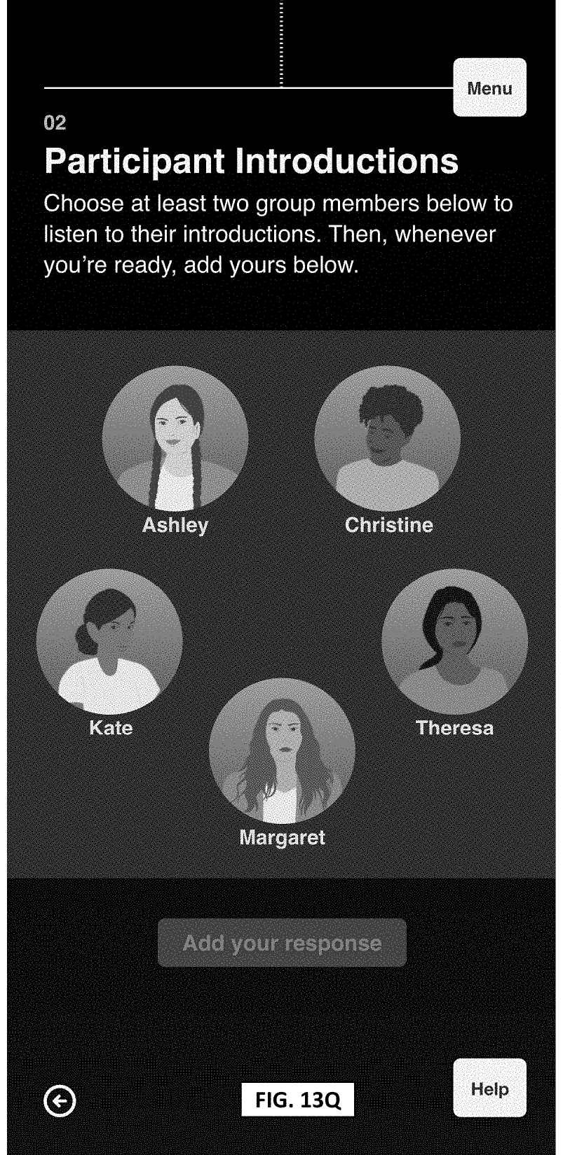

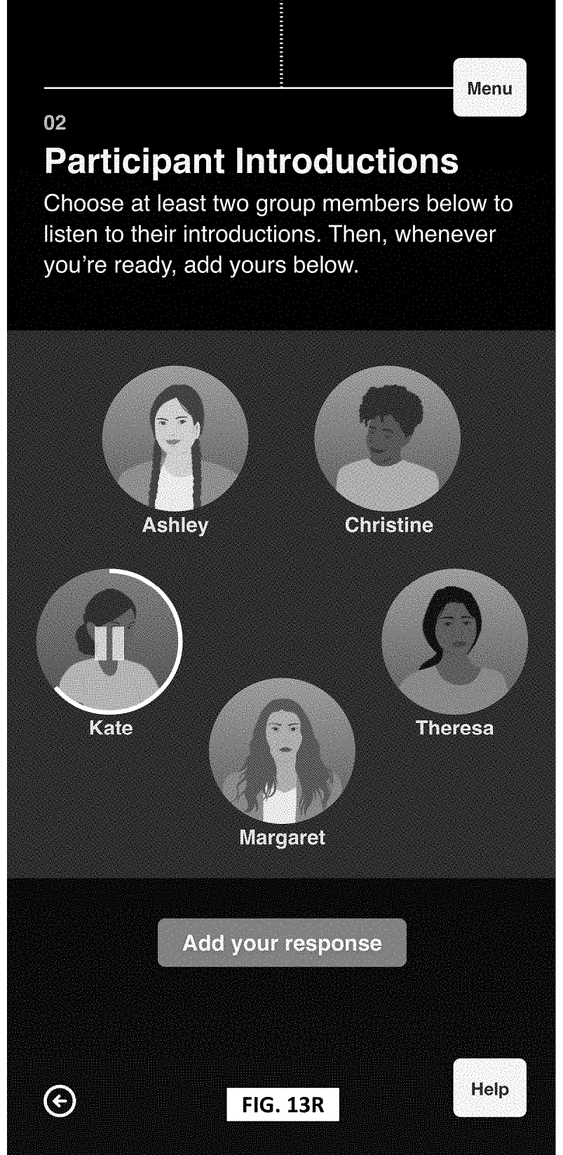

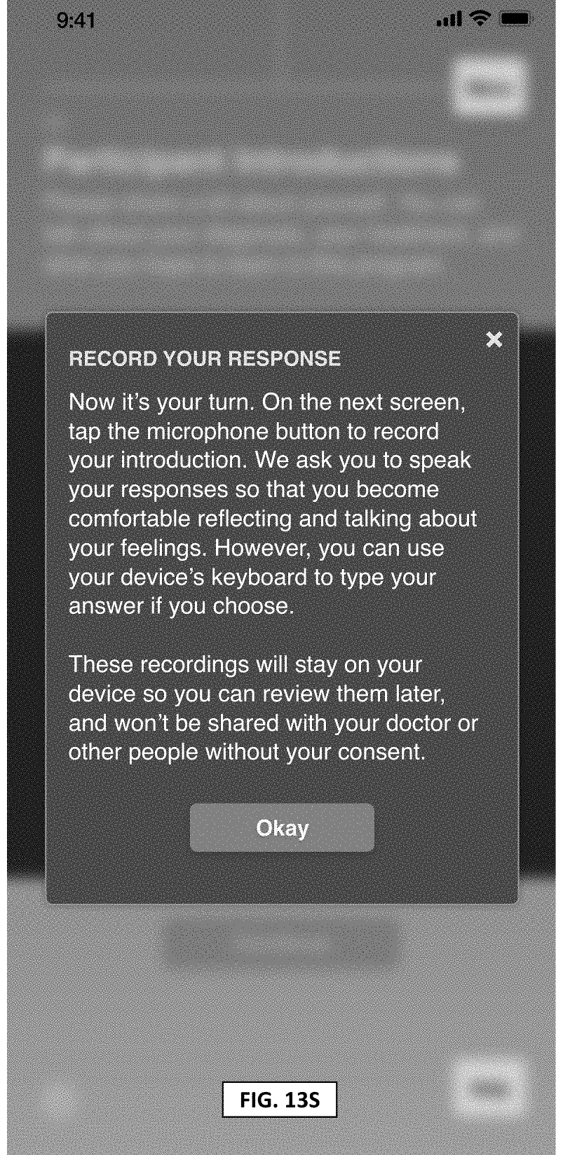

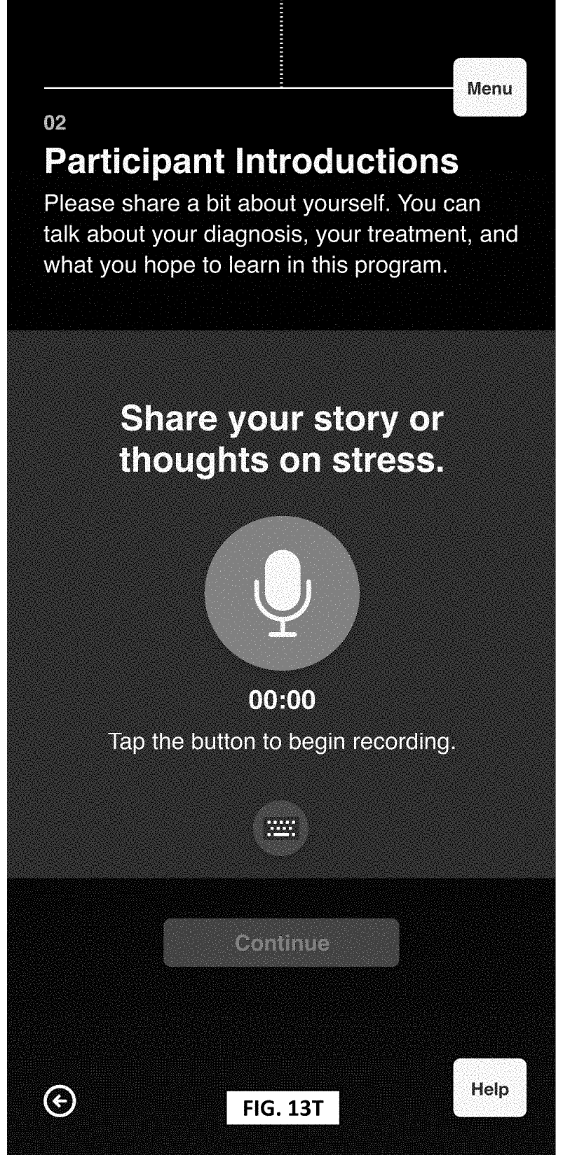

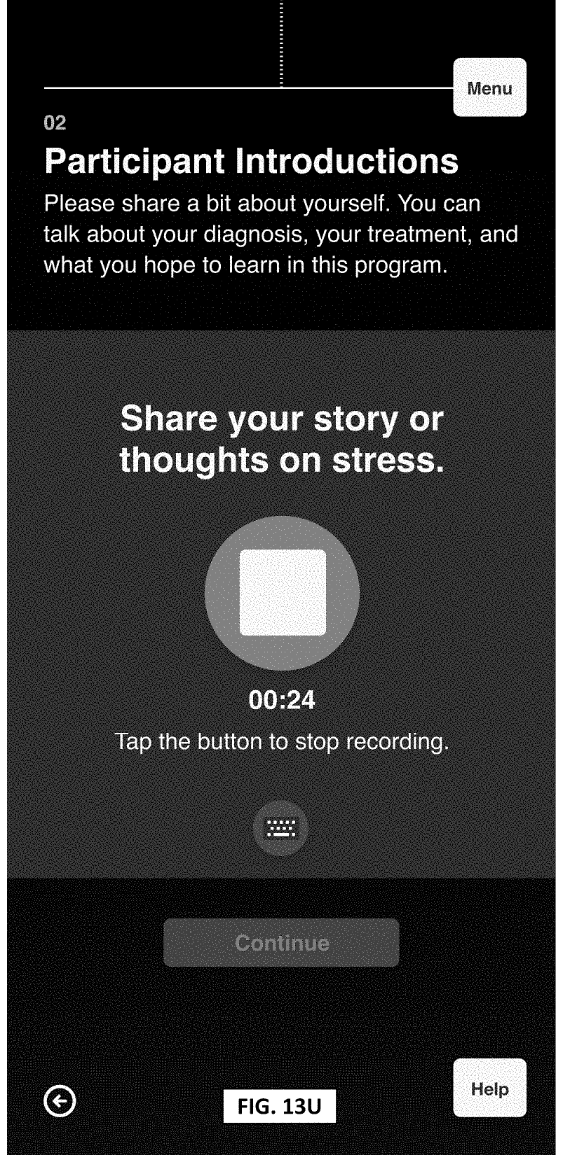

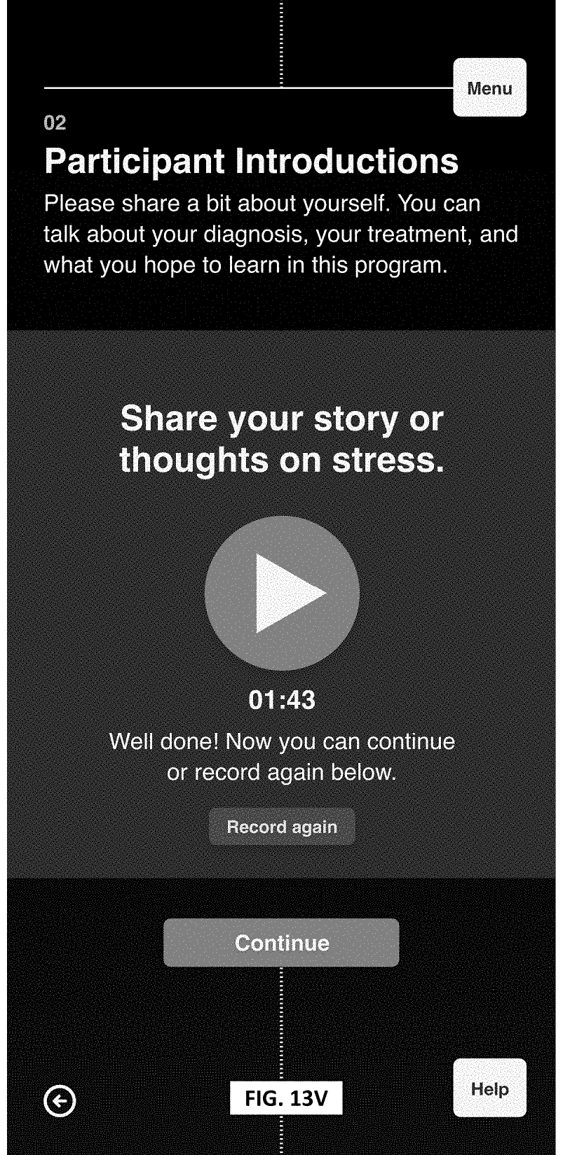

Referring now to FIGS. 13A-13V, therein illustrated are example GUIs implementing aspects of FIGS. 1-2, described above. The GUIs may be presented, for example, on a mobile device, computer, or tablet, among other devices. Aspects of the present disclosure may be implemented using hardware, software, or a combination thereof and can be implemented in one or more computer systems or other processing systems. In one aspect, the disclosure is directed toward one or more computer systems capable of carrying out the functionality described herein. An example of such a computer system 300 is shown in FIG. 3.

FIG. 3 presents an example system diagram of various hardware components and other features, for use in accordance with an aspect of the present disclosure. Aspects of the present disclosure can be implemented using hardware, software, or a combination thereof and can be implemented in one or more computer systems or other processing systems. In one example variation, aspects described herein can be directed toward one or more computer systems capable of carrying out the functionality described herein. An example of such a computer system 300 is shown in FIG. 3.

Computer system 300 includes one or more processors, such as processor 304. The processor 304 is connected to a communication infrastructure 306 (e.g., a communications bus, cross-over bar, or network). In one example, processor 120 can include processor 304. Various software aspects are described in terms of this example computer system. After reading this description, it will become apparent to a person skilled in the relevant art(s) how to implement aspects described herein using other computer systems and/or architectures.

Computer system 300 can include a display interface 302 that forwards graphics, text, and other data from the communication infrastructure 306 (or from a frame buffer not shown) for display on a display unit 330. Computer system 300 also includes a main memory 308, preferably random access memory (RAM), and can also include a secondary memory 310. The secondary memory 310 can include, for example, a hard disk drive 312 and/or a removable storage drive 314, representing a floppy disk drive, a magnetic tape drive, an optical disk drive, etc. The removable storage drive 314 reads from and/or writes to a removable storage unit 318 in a well-known manner. Removable storage unit 318, represents a floppy disk, magnetic tape, optical disk, etc., which is read by and written to removable storage drive 314. As will be appreciated, the removable storage unit 318 includes a computer usable storage medium having stored therein computer software and/or data.

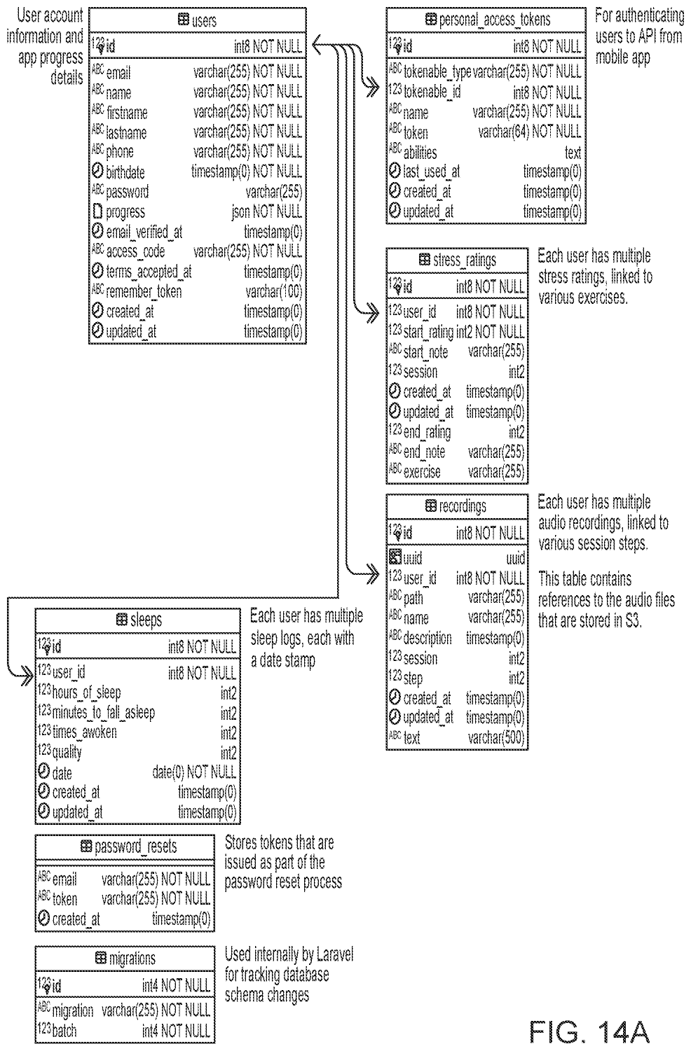

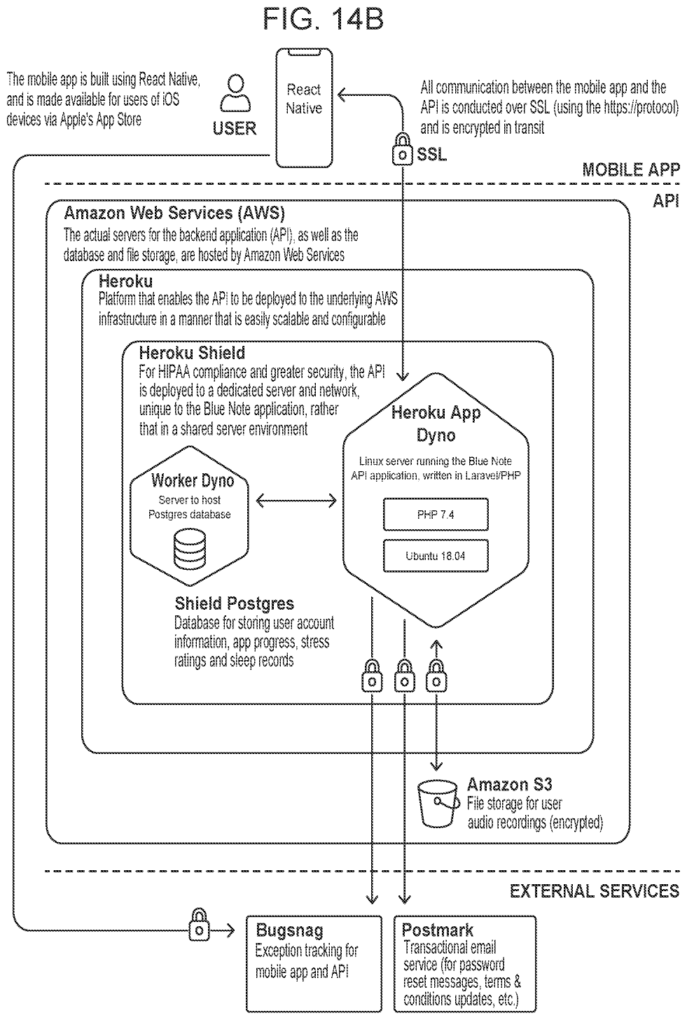

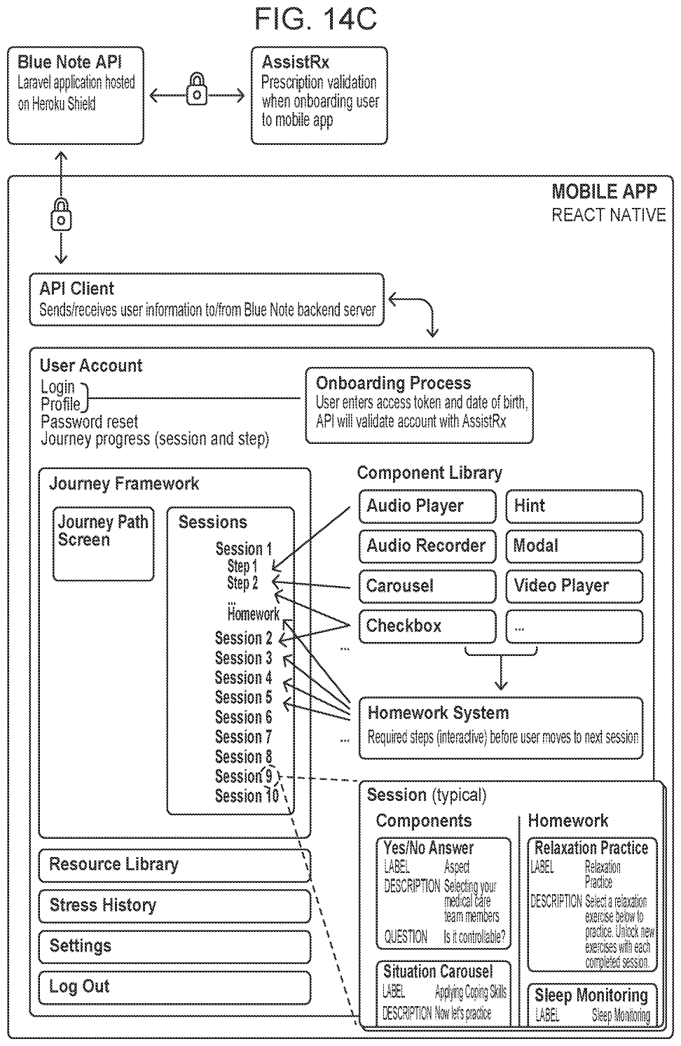

In alternative aspects, secondary memory 310 can include other similar devices for allowing computer programs or other instructions to be loaded into computer system 300. Such devices can include, for example, a removable storage unit 322 and an interface 320. Examples of such can include a program cartridge and cartridge interface (such as that found in video game devices), a removable memory chip (such as an erasable programmable read only memory (EPROM), or programmable read only memory (PROM)) and associated socket, and other removable storage units 322 and interfaces 320, which allow software and data to be transferred from the removable storage unit 322 to computer system 300. Examples of backend schematics are illustrated and described in relation to FIGS. 14A-14C in accordance with aspects of the disclosure.

Computer system 300 can also include a communications interface 324. Communications interface 324 allows software and data to be transferred between computer system 300 and external devices. Examples of communications interface 324 can include a modem, a network interface (such as an Ethernet card), a communications port, a Personal Computer Memory Card International Association (PCMCIA) slot and card, etc. Software and data transferred via communications interface 324 are in the form of signals 328, which can be electronic, electromagnetic, optical or other signals capable of being received by communications interface 324. These signals 328 are provided to communications interface 324 via a communications path (e.g., channel) 326. This path 326 carries signals 328 and can be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 380, a hard disk installed in hard disk drive 370, and signals 328. These computer program products provide software to the computer system 300. Aspects described herein can be directed to such computer program products.

Computer programs (also referred to as computer control logic) are stored in main memory 308 and/or secondary memory 310. Computer programs can also be received via communications interface 324. Such computer programs, when executed, enable the computer system 300 to perform various features in accordance with aspects described herein. In particular, the computer programs, when executed, enable the processor 304 to perform such features. Accordingly, such computer programs represent controllers of the computer system 300.

In variations where aspects described herein are implemented using software, the software can be stored in a computer program product and loaded into computer system 300 using removable storage drive 314, hard disk drive 312, or communications interface 320. The control logic (software), when executed by the processor 304, causes the processor 304 to perform the functions in accordance with aspects described herein as described herein. In another variation, aspects are implemented primarily in hardware using, for example, hardware components, such as application specific integrated circuits (ASICs). Implementation of the hardware state machine so as to perform the functions described herein will be apparent to persons skilled in the relevant art(s).

In yet another example variation, aspects described herein are implemented using a combination of both hardware and software.

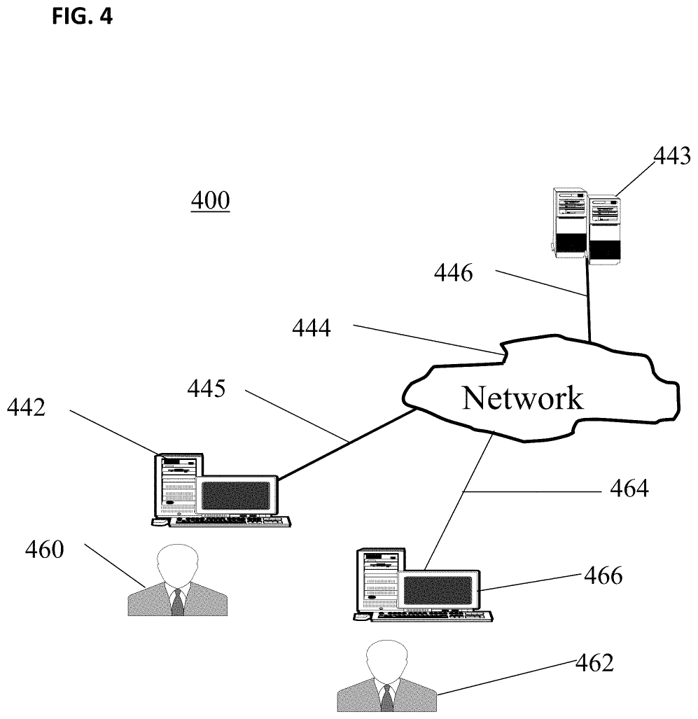

FIG. 4 is a block diagram of various example system components, in accordance with an aspect. FIG. 4 shows a communication system 400 usable in accordance with various aspects described herein. The communication system 400 includes one or more accessors 460, 462 (also referred to interchangeably herein as one or more “users”) and one or more terminals 442, 466. For example, terminals 442, 466 may include vehicle 102 or a related system (e.g., vehicle communication system 110, processor 120, communications device 124), remote device 104, and/or the like. In one aspect, data for use in accordance with aspects described herein is, for example, input and/or accessed by accessors 460, 462 via terminals 442, 466, such as personal computers (PCs), minicomputers, mainframe computers, microcomputers, telephonic devices, or wireless devices, such as personal digital assistants (“PDAs”) or a hand-held wireless devices coupled to a server 443, such as a PC, minicomputer, mainframe computer, microcomputer, or other device having a processor and a repository for data and/or connection to a repository for data, via, for example, a network 444, such as the Internet or an intranet, and couplings 445, 446, 464. The couplings 445, 446, 1464 include, for example, wired, wireless, or fiberoptic links. In another example variation, the method and system in accordance with aspects described herein operate in a stand-alone environment, such as on a single terminal.

The aspects discussed herein can also be described and implemented in the context of computer-readable storage medium storing computer-executable instructions. Computer-readable storage media includes computer storage media and communication media. For example, flash memory drives, digital versatile discs (DVDs), compact discs (CDs), floppy disks, and tape cassettes. Computer-readable storage media can include volatile and nonvolatile, removable and non-removable media implemented in any method or technology for storage of information such as computer readable instructions, data structures, modules or other data.

It will be appreciated that various implementations of the above-disclosed and other features and functions, or alternatives or varieties thereof, can be desirably combined into many other different systems or applications. Also that various presently unforeseen or unanticipated alternatives, modifications, variations, or improvements therein can be subsequently made by those skilled in the art which are also intended to be encompassed by the following claims.

What is claimed is:

1. A computer-implemented method for pre-therapeutic processing comprising:
establishing a baseline psychosocial wellness score for a patient;
establishing an interface between the patient and a digital therapist;
identifying changes from the baseline;
determining, by the digital therapist, based on the identified changes, whether a communication initiation threshold is met;
when the communication initiation threshold is met, receiving an election to participate in assessment from the patient;
formatting, by the digital therapist, patient data, including the changes from the baseline, and assessment for submission;
submitting patient data to a healthcare provider (“HCP”); and
establishing a communication between the patient and the HCP.

2. The computer-implemented method of claim 1, wherein the established communication between the patient and the HCP further comprises receiving, from the HCP, an evaluation of the patient wellness score.

3. The computer-implemented method of claim 2, wherein the established communication between the patient and the HCP further comprises receiving, from the HCP, a clinical judgement and therapeutic decision to treat the patient.

4. The computer-implemented method of claim 3, wherein the established communication between the patient and the HCP further includes at least one of discussing a treatment, therapeutic options, and an execution of a clinical decision.

5. The computer-implemented method of claim 1, wherein the a baseline psychosocial wellness score is established based on at least one of a Beck-Depression Inventory (BDI), a National Comprehensive Cancer Network (NCCN) Distress Thermometer, a Cancer Coping Questionnaire (CCQ), a Cancer Support Source™ (CSS), Hamilton Anxiety Rating Scale (HAM-A), a Hamilton Depression Rating Scale (HAM-D), a Patient-Reported Outcomes Measurement Information System (PROMIS) 57, a Measure of Current Status (MOCS), an Intrusive Events Scale (IES), and a Patient Activation Measure (PAM).

6. A computer-implemented method for therapeutic processing comprising:
receiving, from a patient, a decision to initiate therapy;
establishing an interface between the patient and a digital therapist;
evaluating, by the digital therapist, patient goals;
receiving, by the digital therapist, patient feedback;
initiating therapy by the digital therapist;
formatting and integration of behavioral techniques by the digital therapist;
establishing middle stage of therapy by the digital therapist; and
establishing end stage of therapy by the digital therapist.

7. The computer-implemented method of claim 6, wherein the therapeutic processing further comprises establishing an agenda for the patient.

8. The computer-implemented method of claim 7, wherein the therapeutic processing further comprises receiving, by the digital therapist, second patient feedback regarding the agenda.

9. The computer-implemented method of claim 8, wherein the therapeutic processing is adjusted based upon the patient feedback and the second patient feedback.

10. The computer-implemented method of claim 6, wherein the therapeutic processing is adjusted based upon the patient feedback.

11. The computer-implemented method of claim 6, wherein the patient feedback includes at least one a sleep log, an opinion regarding the therapy, and stress factors.

12. The computer-implemented method of claim 6, wherein the therapy by the digital therapist includes at least one of Cognitive Behavior Therapy (CBT), Relaxation Therapy (RT), Problem-Solving Therapy (PST), Acceptance and Commitment Therapy (ACT), Cognitive Bias Modification (CBM), AIM, and Meta-cognitive Therapy (MCT).

13. A system for therapeutic processing comprising:
a memory; and
a processor coupled with the memory and configured to:
establish a baseline psychosocial wellness score for a patient;
establish an interface between the patient and a digital therapist;
identify changes from the baseline;
determine, based on the identified changes, whether a communication initiation threshold is met;
when the communication initiation threshold is met, receiving an election to participate in assessment from the patient;
format, patient data, including the changes from the baseline, and assessment for submission;
submit patient data to a healthcare provider (“HCP”); and
establish a communication between the patient and the HCP.

14. The system of claim 13, wherein the established communication between the patient and the HCP further comprises receiving, from the HCP, an evaluation of the patient wellness score.

15. The system of claim 14, wherein the established communication between the patient and the HCP further comprises receiving, from the HCP, a clinical judgement and therapeutic decision to treat the patient.

16. The system of claim 15, wherein the established communication between the patient and the HCP further includes at least one of discussing a treatment, therapeutic options, and an execution of a clinical decision.

17. The system of claim 13, wherein the a baseline psychosocial wellness score is established based on at least one of a Beck-Depression Inventory (BDI), a National Comprehensive Cancer Network (NCCN) Distress Thermometer, a Cancer Coping Questionnaire (CCQ), a Cancer Support Source™ (CSS), Hamilton Anxiety Rating Scale (HAM-A), a Hamilton Depression Rating Scale (HAM-D), a Patient-Reported Outcomes Measurement Information System (PROMIS) 57, a Measure of Current Status (MOCS), an Intrusive Events Scale (IES), and a Patient Activation Measure (PAM).
